(19) United States
(12) Patent Application Publication
(10) Pub. No.: US 2023/0303598 A1
(43) Pub. Date: Sep. 28, 2023

(54) BIOINSPIRED LIPID DERIVATIVES AND USES THEREOF

(71) Applicant: Ohio State Innovation Foundation, Columbus, OH (US)

(72) Inventors: Yizhou DONG, Dublin, OH (US); Yuebao ZHANG, Columbus, OH (US)

(21) Appl. No.: 18/022,017

(22) PCT Filed: Aug. 18, 2021
(86) PCT No.: PCT/US2021/046501

§ 371 Date: Feb. 17, 2023

Related U.S. Application Data
(60) Provisional application No. 63/067,030, filed on Aug. 18, 2020.

(51) Int. Cl. C07F 9/24; C07F 9/40; C07F 9/655; C07F 9/10; A61K 9/51

(52) U.S. Cl. CPC C07F 9/2458; C07F 9/4075; C07F 9/65515; C07F 9/10; A61K 9/5123; A61K 9/5146

(57) ABSTRACT

The present disclosure relates to compounds, compositions, and methods for delivery of therapeutic, diagnostic, or prophylactic agents (for example, a nucleic acid).

### CROSS-REFERENCE TO RELATED APPLICATIONS

The application claims the benefit of U.S. Provisional Application No. 63/067,030, filed Aug. 18, 2020, which is hereby incorporated herein by reference in its entirety.

### STATEMENT REGARDING FEDERALLY SPONSORED RESEARCH

This invention was made with government support under Grant No. R35GM119679 awarded by the National Institutes of Health. The Government has certain rights in the invention.

### FIELD

The present disclosure relates to compounds, compositions, and methods for delivery of therapeutic, diagnostic, or prophylactic agents (for example, a nucleic acid).

### BACKGROUND

Messenger RNA (mRNA) based therapeutics have shown great promise for expressing functional antibodies and proteins. Clinical studies have explored mRNA for use in gene therapy and immunotherapy as well as in vaccines. Efficient delivery of mRNA is a key step and challenge for mRNA therapeutics. Despite promising data from ongoing clinical trials, the clinical use of mRNA requires the discovery and development of more efficient delivery systems.

The compounds, compositions, and methods disclosed herein address these and other needs.

### SUMMARY

The present disclosure provides compounds, compositions, and methods of use thereof. Also provided are compositions including a compound of the invention and an agent (e.g., an mRNA). The present disclosure also provides methods of using the compositions for delivering an agent to a subject.

In one aspect, the disclosure provides a compound of Formula A:

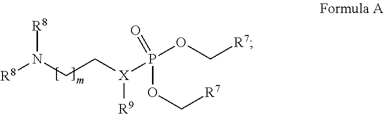

or a salt thereof,
wherein:
each R⁷ is independently selected from alkyl, alkenyl, alkynyl, ester, alkylester, alkylketal, alkylcarbonate, or alkylcarbamate;
each R8 is independently selected from alkyl, alkenyl, alkynyl, ester, alkylester, or alkylalcohol;
R⁹ is selected from hydrogen, alkyl, alkenyl, alkynyl, ester, alkylester, or

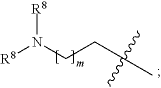

X is selected from O or N; and
m is an integer from 0 to 10.

In some embodiments, each R⁸ is alkyl. In some embodiments, each R⁸ is methyl. In some embodiments, each R⁸ is alkylalcohol.

In some embodiments, R⁹ is hydrogen. In some embodiments, R⁹ is

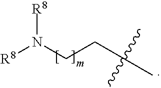

In some embodiments, m is an integer from 1 to 3. In some embodiments, X is O. In some embodiments, X is N, and R⁹ is hydrogen. In some embodiments, X is N, and R⁹ is

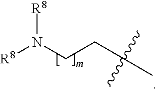

In one aspect, the disclosure provides a compound of Formula I:

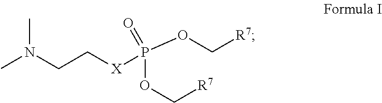

or a salt thereof,
wherein:
each R⁷ is independently selected from alkyl, alkenyl, alkynyl, ester, alkylester, alkylketal, alkylcarbonate, or alkylcarbamate; and
X is selected from O or N.

In one aspect, the disclosure provides a compound of Formula II:

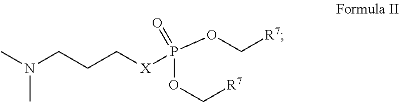

or a salt thereof,
wherein:
each R⁷ is independently selected from alkyl, alkenyl, alkynyl, ester, alkylester, alkylketal, alkylcarbonate, or alkylcarbamate; and
X is selected from O or N.

In one aspect, the disclosure provides a compound of Formula III:

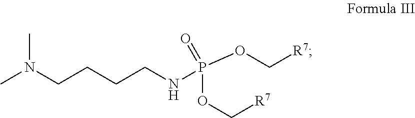

or a salt thereof,
wherein:
each R⁷ is independently selected from alkyl, alkenyl, alkynyl, ester, alkylester, alkylketal, alkylcarbonate, or alkylcarbamate.

In one aspect, the disclosure provides a compound of Formula IV:

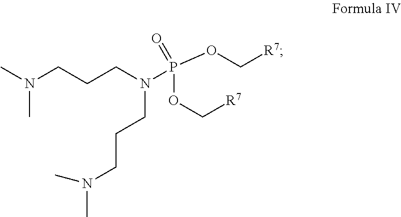

or a salt thereof,
wherein:
each R⁷ is independently selected from alkyl, alkenyl, alkynyl, ester, alkylester, alkylketal, alkylcarbonate, or alkylcarbamate.

In one aspect, the disclosure provides a compound of Formula V:

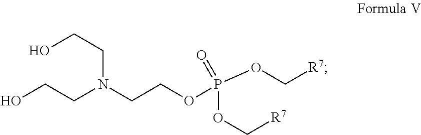

or a salt thereof,
wherein:
each R⁷ is independently selected from alkyl, alkenyl, alkynyl, ester, alkylester, alkylketal, alkylcarbonate, or alkylcarbamate.

In one aspect, the disclosure provides a compound of Formula VI:

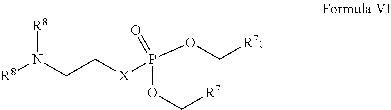

or a salt thereof,
wherein:
each R⁷ is independently selected from alkyl, alkenyl, alkynyl, ester, alkylester, alkylketal, alkylcarbonate, or alkylcarbamate;
each R⁸ is independently selected from alkyl, alkenyl, alkynyl, ester, alkylester, or alkylalcohol; and
X is selected from O or N.

In one aspect, the disclosure provides a compound of Formula VII:

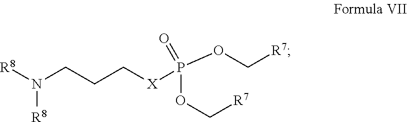

or a salt thereof,
wherein:
each R⁷ is independently selected from alkyl, alkenyl, alkynyl, ester, alkylester, alkylketal, alkylcarbonate, or alkylcarbamate;
each R⁸ is independently selected from alkyl, alkenyl, alkynyl, ester, alkylester, or alkylalcohol; and
X is selected from O or N.

In one aspect, the disclosure provides a compound of Formula VIII:

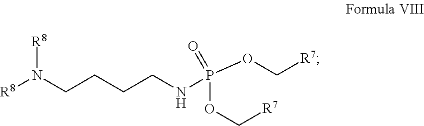

or a salt thereof,
wherein:
each R⁷ is independently selected from alkyl, alkenyl, alkynyl, ester, alkylester, alkylketal, alkylcarbonate, or alkylcarbamate; and
each R⁸ is independently selected from alkyl, alkenyl, alkynyl, ester, alkylester, or alkylalcohol.

In one aspect, the disclosure provides a compound of Formula IX:

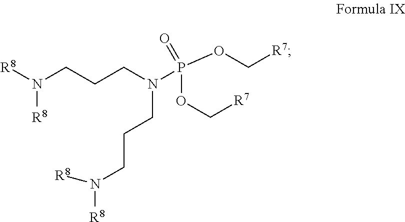

or a salt thereof,
wherein:
each R⁷ is independently selected from alkyl, alkenyl, alkynyl, ester, alkylester, alkylketal, alkylcarbonate, or alkylcarbamate; and
each R⁸ is independently selected from alkyl, alkenyl, alkynyl, ester, alkylester, or alkylalcohol.

In one aspect, the disclosure provides a compound of Formula X:

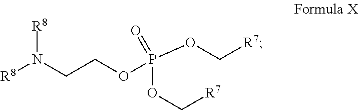

or a salt thereof,
wherein:
each R⁷ is independently selected from alkyl, alkenyl, alkynyl, ester, alkylester, alkylketal, alkylcarbonate, or alkylcarbamate; and
each R⁸ is independently selected from alkyl, alkenyl, alkynyl, ester, alkylester, or alkylalcohol.

In some embodiments, each R⁷ is independently selected from:

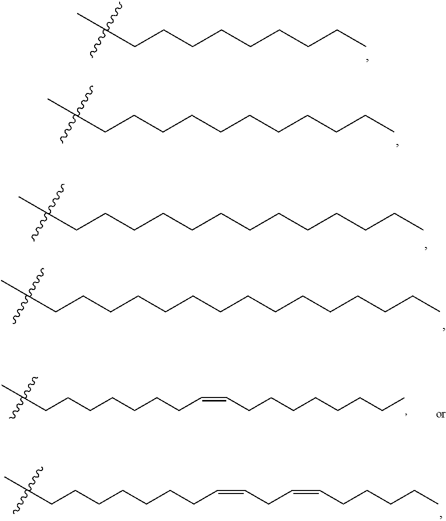

In some embodiments, the compound has the formula:

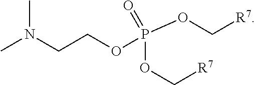

wherein each R⁷ is independently selected from:

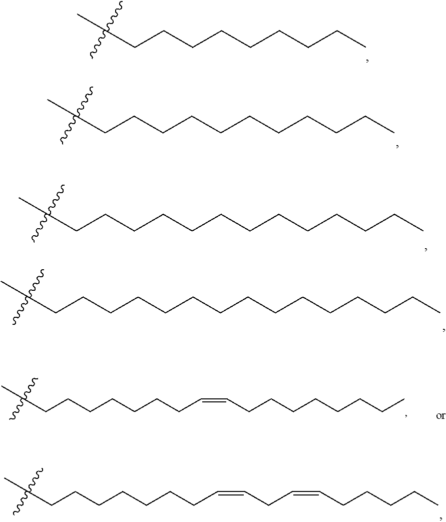

In some embodiments the compound has the formula:

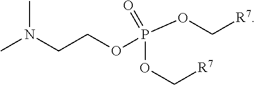

wherein each R⁷ is:

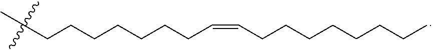 [SMILES: *CCCCCCC/C=C\CCCCCCCC]

In some embodiments the compound has the formula:

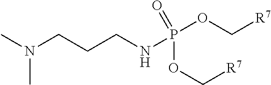

wherein each R⁷ is:

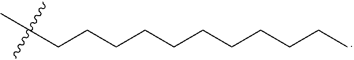 [SMILES: *CCCCCCCCCCC]

In some embodiments, the disclosure provides a composition comprising:
a compound of Formula A; and
an agent.

In some embodiments, the disclosure provides a composition comprising:
a compound of Formula I, II, III, IV, or V; and
an agent.

In some embodiments, the disclosure provides a composition comprising:
a compound of Formula VI, VII, VIII, IX, or X; and
an agent.

In some embodiments, the disclosure provides a nanoparticle comprising:
a compound of Formula A;
a non-cationic lipid;
a polyethylene glycol-lipid; and
a sterol.

In some embodiments, the disclosure provides a nanoparticle comprising:
a compound of Formula I, II, III, IV, or V;
a non-cationic lipid;
a polyethylene glycol-lipid; and
a sterol.

In some embodiments, the disclosure provides a nanoparticle comprising:
a compound of Formula VI, VII, VIII, IX, or X;
a non-cationic lipid;
a polyethylene glycol-lipid; and
a sterol.

In some embodiments, the nanoparticle further comprises an agent.

In some embodiments, the agent is a polynucleotide. In some embodiments, the agent is an RNA. In some embodiments, the agent is an mRNA.

In some embodiments, disclosed herein is a pharmaceutical composition comprising a pharmaceutically acceptable carrier and a therapeutically effective amount of a compound of Formula A, I, II, III, IV, V, VI, VII, VIII, IX, or X.

In some embodiments, provided are methods for the delivery of an agent into a cell. In some embodiments, provided are methods for the delivery of nucleic acids. In some embodiments, provided herein are methods for the delivery of polynucleotides.

### BRIEF DESCRIPTION OF THE DRAWINGS

The accompanying figures, which are incorporated in and constitute a part of this specification, illustrate several aspects described below.

FIG. 1 illustrates the results of in vitro mRNA delivery screening of ionizable lipids in Hep3b cells at a ratio of 20:30:40:0.75 (compound/DOPE/cholesterol/PEG-lipid).

FIG. 2 illustrates the results of in vitro mRNA delivery screening of ionizable lipids in Hep3b cells at a ratio of 40:30:40:0.75 (compound/DOPE/cholesterol/PEG-lipid).

FIG. 3 illustrates the results of in vitro mRNA delivery screening of ionizable lipids in Hep3b cells at a ratio of 60:30:40:0.75 (compound/DOPE/cholesterol/PEG-lipid).

### DETAILED DESCRIPTION

The present disclosure provides new compounds, bioinspired lipid derivatives, compositions, nanoparticles, and methods of use thereof. Also provided are compositions including a compound of the invention and an agent (e.g., an mRNA). The present disclosure also provides methods using the compositions for delivering an agent to a cell or to a subject.

These bioinspired compounds are used in applications such as gene therapy, drug delivery, and vaccines.

Reference will now be made in detail to the embodiments of the invention, examples of which are illustrated in the drawings and the examples. This invention may, however, be embodied in many different forms and should not be construed as limited to the embodiments set forth herein. Unless defined otherwise, all technical and scientific terms used herein have the same meaning as commonly understood to one of ordinary skill in the art to which this invention belongs. The following definitions are provided for the full understanding of terms used in this specification.

### Definitions

General Definitions

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. The following definitions are provided for the full understanding of terms used in this specification.

As used herein, the article “a,” “an,” and “the” means “at least one,” unless the context in which the article is used clearly indicates otherwise.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “promoter” or “regulatory element” refers to a region or sequence determinants located upstream or downstream from the start of transcription and which are involved in recognition and binding of RNA polymerase and other proteins to initiate transcription.

Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 0-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,⁹⁴%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information (http://www.ncbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

The term “about” as used herein when referring to a measurable value such as an amount, a percentage, and the like, is meant to encompass variations of 20%, ±10%, +5%, or +1% from the measurable value.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

As used herein, the term “controlled-release” or “controlled-release drug delivery” or “extended release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

The phrases “concurrent administration”, “administration in combination”, “simultaneous administration” or “administered simultaneously” as used herein, means that the compounds are administered at the same point in time or immediately following one another.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain.

Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. For example, fragments of antibodies which maintain binding activity are included within the meaning of the term “antibody or antigen binding fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as—OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula C(O)H. Throughout this specification “C(O)” or “CO” is a short hand notation for C═O.

The terms “amine” or “amino” as used herein are represented by the formula—NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula—C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —S¹Z¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “phosphonyl” is used herein to refer to the phospho-oxo group represented by the formula —P(O)(OZ¹)₂, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

The term “thio” as used herein is represented by the formula —S—.

“R¹,” “R2,” “R3,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxyl group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

Compounds

In one aspect, the disclosure provides a compound of Formula A:

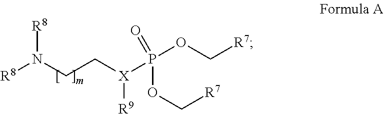

or a salt thereof,
wherein:
each R⁷ is independently selected from alkyl, alkenyl, alkynyl, ester, alkylester, alkylketal, alkylcarbonate, or alkylcarbamate;
each R⁸ is independently selected from alkyl, alkenyl, alkynyl, ester, alkylester, or alkylalcohol;
R⁹ is selected from hydrogen, alkyl, alkenyl, alkynyl, ester, alkylester, or

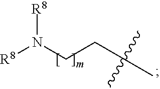

X is selected from O or N; and
m is an integer from 0 to 10.

In some embodiments, each R⁸ is alkyl. In some embodiments, each R⁸ is methyl. In some embodiments, each R⁸ is alkylalcohol.

In some embodiments, R⁹ is hydrogen. In some embodiments, R⁹ is R⁸

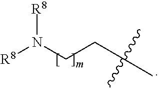

In some embodiments, m is an integer from 1 to 3. In some embodiments, X is O. In some embodiments, X is N, and R⁹ is hydrogen. In some embodiments, X is N, and R⁹ is

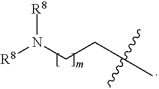

In one aspect, the disclosure provides a compound of Formula I:

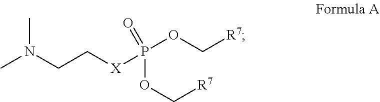

or a salt thereof,
wherein:
each R⁷ is independently selected from alkyl, alkenyl, alkynyl, ester, alkylester, alkylketal, alkylcarbonate, or alkylcarbamate; and
X is selected from O or N.

In one aspect, the disclosure provides a compound of Formula II:

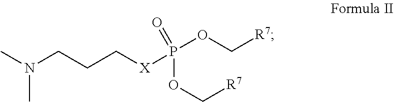

or a salt thereof,
wherein:
each R⁷ is independently selected from alkyl, alkenyl, alkynyl, ester, alkylester, alkylketal, alkylcarbonate, or alkylcarbamate; and
X is selected from O or N.

In one aspect, the disclosure provides a compound of Formula III:

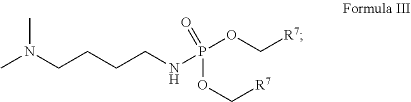

or a salt thereof,
wherein:
each R⁷ is independently selected from alkyl, alkenyl, alkynyl, ester, alkylester, alkylketal, alkylcarbonate, or alkylcarbamate.

In one aspect, the disclosure provides a compound of Formula IV:

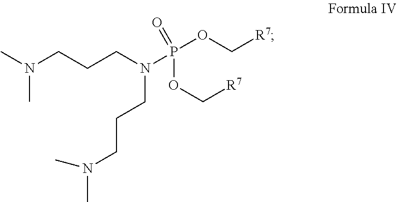

or a salt thereof,
wherein:
each R⁷ is independently selected from alkyl, alkenyl, alkynyl, ester, alkylester, alkylketal, alkylcarbonate, or alkylcarbamate.

In one aspect, the disclosure provides a compound of Formula V:

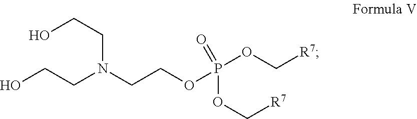

or a salt thereof,
wherein:
each R⁷ is independently selected from alkyl, alkenyl, alkynyl, ester, alkylester, alkylketal, alkylcarbonate, or alkylcarbamate.

In one aspect, the disclosure provides a compound of Formula VI:

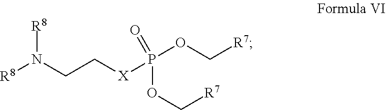

or a salt thereof,
wherein:
each R⁷ is independently selected from alkyl, alkenyl, alkynyl, ester, alkylester, alkylketal, alkylcarbonate, or alkylcarbamate;
each R⁸ is independently selected from alkyl, alkenyl, alkynyl, ester, alkylester, or alkylalcohol; and
X is selected from O or N.

In one aspect, the disclosure provides a compound of Formula VII:

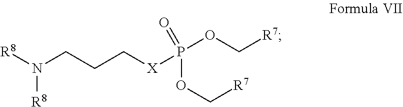

or a salt thereof,
wherein:
each R⁷ is independently selected from alkyl, alkenyl, alkynyl, ester, alkylester, alkylketal, alkylcarbonate, or alkylcarbamate;
each R⁸ is independently selected from alkyl, alkenyl, alkynyl, ester, alkylester, or alkylalcohol; and
X is selected from O or N.

In one aspect, the disclosure provides a compound of Formula VIII:

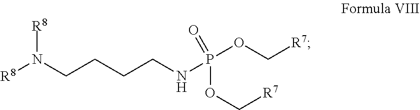

or a salt thereof,
wherein:
each R⁷ is independently selected from alkyl, alkenyl, alkynyl, ester, alkylester, alkylketal, alkylcarbonate, or alkylcarbamate; and
each R⁸ is independently selected from alkyl, alkenyl, alkynyl, ester, alkylester, or alkylalcohol.

In one aspect, the disclosure provides a compound of Formula IX:

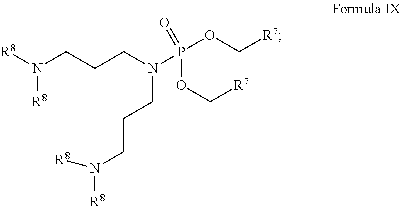

or a salt thereof,
wherein:
each R⁷ is independently selected from alkyl, alkenyl, alkynyl, ester, alkylester, alkylketal, alkylcarbonate, or alkylcarbamate; and
each R⁸ is independently selected from alkyl, alkenyl, alkynyl, ester, alkylester, or alkylalcohol.

In one aspect, the disclosure provides a compound of Formula X:

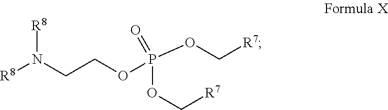

or a salt thereof,
wherein:
each R⁷ is independently selected from alkyl, alkenyl, alkynyl, ester, alkylester, alkylketal, alkylcarbonate, or alkylcarbamate; and
each R⁸ is independently selected from alkyl, alkenyl, alkynyl, ester, alkylester, or alkylalcohol.

In some embodiments, each R⁷ is independently selected from:

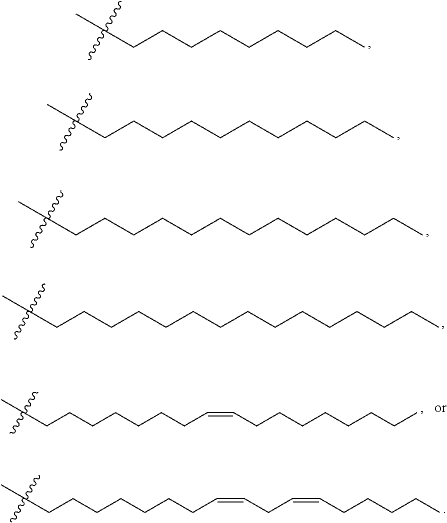

In some embodiments, the compound has the formula:

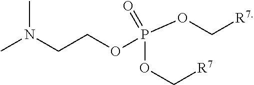

wherein each R⁷ is independently selected from:

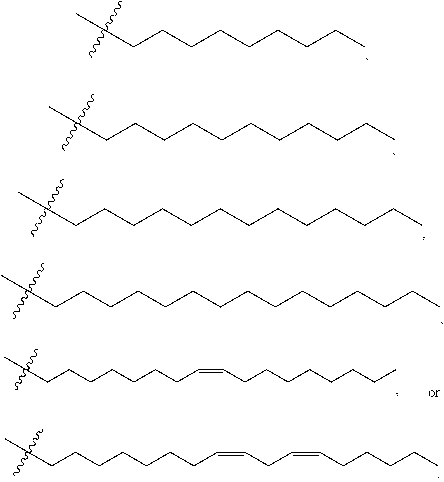

In some embodiments, the compound has the formula:

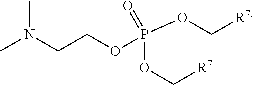

wherein each R⁷ is:

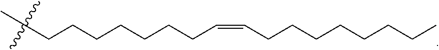 [SMILES: *CCCCCCC/C=C\CCCCCCCC]

In some embodiments, the compound has the formula:

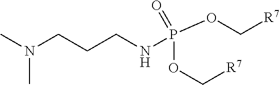

wherein each R⁷ is:

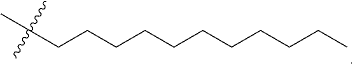 [SMILES: *CCCCCCCCCCC]

In some embodiments, each R⁷ is independently selected from alkyl, alkenyl, alkynyl, ester, alkylester, alkylketal, alkylcarbonate, or alkylcarbamate. In some embodiments, each R⁷ is selected from alkyl, alkenyl, alkynyl, ester, or alkylester. In some embodiments, each R⁷ is selected from alkyl or alkenyl. In some embodiments, each R⁷ is alkyl. In some embodiments, each R⁷ is alkenyl. In some embodiments, each R⁷ is alkynyl. In some embodiments, each R⁷ is ester. In some embodiments, each R⁷ is alkylester.

In some embodiments, each R⁷ is C₇₋₁₇alkyl. In some embodiments, each R⁷ is C₇alkyl. In some embodiments, each R⁷ is C₈alkyl. In some embodiments, each R⁷ is C₉alkyl. In some embodiments, each R⁷ is C₁₀alkyl. In some embodiments, each R⁷ is C₁₁alkyl. In some embodiments, each R⁷ is C₁₂alkyl. In some embodiments, each R⁷ is C₁₃alkyl. In some embodiments, each R⁷ is C₁₄alkyl. In some embodiments, each R⁷ is C₁₅alkyl. In some embodiments, each R⁷ is C₁₆alkyl. In some embodiments, each R⁷ is C₁₇alkyl.

In some embodiments, each R⁷ is C₁₀₋₂₁alkenyl. In some embodiments, each R⁷ is C₁₀alkenyl. In some embodiments, each R⁷ is C₁₁alkenyl. In some embodiments, each R⁷ is C₁₂alkenyl. In some embodiments, each R⁷ is C₁₃alkenyl. In some embodiments, each R⁷ is C₁₄alkenyl. In some embodiments, each R⁷ is C₁₅alkenyl. In some embodiments, each R⁷ is C₁₆alkenyl. In some embodiments, each R⁷ is C₁₇alkenyl. In some embodiments, each R⁷ is C₁₈alkenyl. In some embodiments, each R⁷ is C₁₉alkenyl. In some embodiments, each R⁷ is C₂₀alkenyl. In some embodiments, each R⁷ is C₂₁alkenyl.

In some embodiments, each R⁷ is

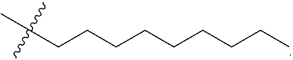 [SMILES: *CCCCCCCCC]

In some embodiments, each R⁷ is

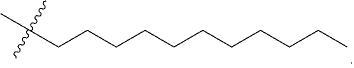 [SMILES: *CCCCCCCCCCC]

In some embodiments, each R⁷ is

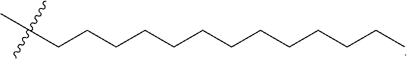 [SMILES: *CCCCCCCCCCCCC]

In some embodiments, each R⁷ is

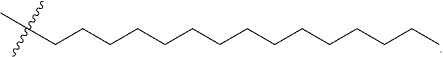 [SMILES: *CCCCCCCCCCCCCCC]

In some embodiments, each R⁷ is

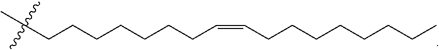 [SMILES: *CCCCCCC/C=C\CCCCCCCC]

In some embodiments, each R⁸ is

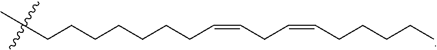 [SMILES: *CCCCCCC/C=C\C/C=C\CCCCC]

In some embodiments, each R⁸ is independently selected from alkyl, alkenyl, alkynyl, ester, alkylester, or alkylalcohol. In some embodiments, each R⁸ is alkyl. In some embodiments, each R⁸ is alkenyl. In some embodiments, each R⁸ is alkynyl. In some embodiments, each R⁸ is ester. In some embodiments, each R⁸ is alkylester. In some embodiments, each R⁸ is alkylalcohol. In some embodiments, each R⁸ is methyl. In some embodiments, each R⁸ is ethyl.

In some embodiments, at least one R⁷ is independently selected from alkyl, alkenyl, alkynyl, ester, alkylester, alkylketal, alkylcarbonate, or alkylcarbamate. In some embodiments, at least one R⁷ is selected from alkyl, alkenyl, alkynyl, ester, or alkylester. In some embodiments, at least one R⁷ is selected from alkyl or alkenyl. In some embodiments, at least one R⁷ is alkyl. In some embodiments, at least one R⁷ is alkenyl. In some embodiments, at least one R⁷ is alkynyl. In some embodiments, at least one R⁷ is ester. In some embodiments, at least one R⁷ is alkylester.

In some embodiments, at least one R⁷ is

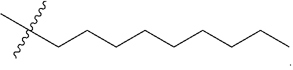 [SMILES: *CCCCCCCCC]

In some embodiments, at least one R⁷ is

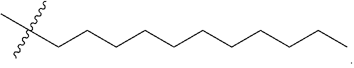 [SMILES: *CCCCCCCCCCC]

In some embodiments, at least one R⁷ is

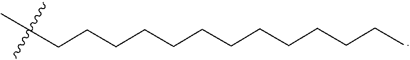 [SMILES: *CCCCCCCCCCCCC]

In some embodiments, at least one R⁷ is

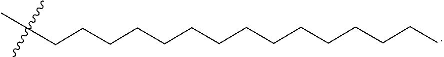 [SMILES: *CCCCCCCCCCCCCCC]

In some embodiments, at least one R⁷ is

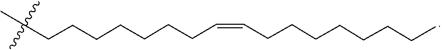 [SMILES: *CCCCCCC/C=C\CCCCCCCC]

In some embodiments, at least one R⁷ is

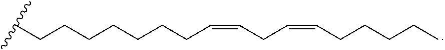 [SMILES: *CCCCCCC/C=C\C/C=C\CCCCC]

In some embodiments, at least one R⁷ is C₇₋₁₇alkyl. In some embodiments, at least one R⁷ is C₇alkyl. In some embodiments, at least one R⁷ is C₈alkyl. In some embodiments, at least one R⁷ is C₉alkyl. In some embodiments, at least one R⁷ is C₁₀alkyl. In some embodiments, at least one R⁷ is C₁₁alkyl. In some embodiments, at least one R⁷ is C₁₂alkyl. In some embodiments, at least one R⁷ is C₁₃alkyl. In some embodiments, at least one R⁷ is C₁₄alkyl. In some embodiments, at least one R⁷ is C₁₅alkyl. In some embodiments, at least one R⁷ is C₁₆alkyl. In some embodiments, at least one R⁷ is C₁₇alkyl.

In some embodiments, at least one R⁷ is C₁₀₋₂₁alkenyl. In some embodiments, at least one R⁷ is C₁₀alkenyl. In some embodiments, at least one R⁷ is C₁₁alkenyl. In some embodiments, at least one R⁷ is C₁₂alkenyl. In some embodiments, at least one R⁷ is C₁₃alkenyl. In some embodiments, at least one R⁷ is C₁₄alkenyl. In some embodiments, at least one R⁷ is C₁₅alkenyl. In some embodiments, at least one R⁷ is C₁₆alkenyl. In some embodiments, at least one R⁷ is C₁₇alkenyl. In some embodiments, at least one R⁷ is C₁₈alkenyl. In some embodiments, at least one R⁷ is C₁₉alkenyl. In some embodiments, at least one R⁷ is C₂₀alkenyl. In some embodiments, at least one R⁷ is C₂₁alkenyl.

In some embodiments, at least one R⁸ is independently selected from alkyl, alkenyl, alkynyl, ester, alkylester, or alkylalcohol. In some embodiments, at least one R⁸ is alkyl. In some embodiments, at least one R⁸ is alkenyl. In some embodiments, at least one R⁸ is alkynyl. In some embodiments, at least one R⁸ is ester. In some embodiments, at least one R⁸ is alkylester. In some embodiments, at least one R⁸ is alkylalcohol. In some embodiments, at least one R⁸ is methyl. In some embodiments, at least one R⁸ is ethyl.

In some embodiments, R⁹ is selected from hydrogen, alkyl, alkenyl, alkynyl, ester, alkylester, or

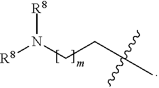

In some embodiments, R⁹ is hydrogen. In some embodiments, R⁹ is alkyl. In some embodiments, R⁹ is alkenyl. In some embodiments, R⁹ is alkynyl. In some embodiments, R⁹ is ester. In some embodiments, R⁹ is alkylester. In some embodiments, R⁹ is

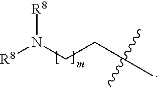

In some embodiments, X is selected from O or N. In some embodiments, X is O. In some embodiments, X is N.

In some embodiments, m is an integer from 0 to 10. In some embodiments, m is an integer from 1 to 3. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

In some embodiments, X is O. In some embodiments, X is N, and R⁹ is hydrogen. In some embodiments, X is N, and R⁹ is

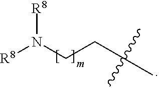

In some embodiments, the alkyl is substituted. In some embodiments, the alkyl is unsubstituted. In some embodiments, the alkenyl is substituted. In some embodiments, the alkenyl is unsubstituted.

Nanoparticles

In one aspect, the disclosure provides a nanoparticle comprising:
a compound of Formula A, I, II, III, IV, V, VI, VII, VIII, IX, or X;
a non-cationic lipid;
a polyethylene glycol-lipid; and
a sterol.

In one aspect, the disclosure provides a nanoparticle comprising: a compound of Formula A; a non-cationic lipid; a polyethylene glycol-lipid; and a sterol.

In one aspect, the disclosure provides a nanoparticle comprising: a compound of Formula I; a non-cationic lipid; a polyethylene glycol-lipid; and a sterol.

In one aspect, the disclosure provides a nanoparticle comprising: a compound of Formula II; a non-cationic lipid; a polyethylene glycol-lipid; and a sterol.

In one aspect, the disclosure provides a nanoparticle comprising: a compound of Formula III; a non-cationic lipid; a polyethylene glycol-lipid; and a sterol.

In one aspect, the disclosure provides a nanoparticle comprising: a compound of Formula IV; a non-cationic lipid; a polyethylene glycol-lipid; and a sterol.

In one aspect, the disclosure provides a nanoparticle comprising: a compound of Formula V; a non-cationic lipid; a polyethylene glycol-lipid; and a sterol.

In one aspect, the disclosure provides a nanoparticle comprising: a compound of Formula VI; a non-cationic lipid; a polyethylene glycol-lipid; and a sterol.

In one aspect, the disclosure provides a nanoparticle comprising: a compound of Formula VII; a non-cationic lipid; a polyethylene glycol-lipid; and a sterol.

In one aspect, the disclosure provides a nanoparticle comprising: a compound of Formula VIII; a non-cationic lipid; a polyethylene glycol-lipid; and a sterol.

In one aspect, the disclosure provides a nanoparticle comprising: a compound of Formula IX; a non-cationic lipid; a polyethylene glycol-lipid; and a sterol.

In one aspect, the disclosure provides a nanoparticle comprising: a compound of Formula X; a non-cationic lipid; a polyethylene glycol-lipid; and a sterol.

The various compounds of Formula A, I, II, III, IV, V, VI, VII, VIII, IX, or X are described in the Compounds section above. In some embodiments, the nanoparticle comprises a compound of Formula A, I, II, III, IV, V, VI, VII, VIII, IX, or X in a molar ratio of about 5% to about 80%. In some embodiments, the nanoparticle comprises a compound of Formula A, I, II, III, IV, V, VI, VII, VIII, IX, or X in a molar ratio of about 20% to about 60%. In some embodiments, the nanoparticle comprises a compound of Formula A, I, II, III, IV, V, VI, VII, VIII, IX, or X in a molar ratio of about 5%, about 10%, about 15%, about 20%, about 25%, about 30%, about 35%, about 40%, about 45%, about 50%, about 55%, about 60%, about 65%, about 70%, about 75%, or about 80%. In one embodiment, the nanoparticle comprises a compound of Formula A, I, II, III, IV, V, VI, VII, VIII, IX, or X in a molar ratio of about 20%. In one embodiment, the nanoparticle comprises a compound of Formula A, I, II, III, IV, V, VI, VII, VIII, IX, or X in a molar ratio of about 40%. In one embodiment, the nanoparticle comprises a compound of Formula A, I, II, III, IV, V, VI, VII, VIII, IX, or X in a molar ratio of about 60%.

In some embodiments, the nanoparticle comprises a non-cationic lipid. In some embodiments, the non-cationic lipid interacts with the lipids as a helper lipid. In some embodiments, the non-cationic lipid can include, but is not limited to, 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), DPPC (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), or combinations thereof. In one embodiment, the non-cationic lipid is 1,2-dioleoyl-sn-glycero-3-phosphoethanolamine (DOPE).

In one embodiment, the non-cationic lipid is 1-palmitoyl-2-oleoyl-sn-glycero-3-phosphoethanolamine (POPE), In one embodiment, the non-cationic lipid is 1,2-distearoyl-sn-glycero-3-phosphocholine (DSPC). In one embodiment, the non-cationic lipid is 1-stearoyl-2-oleoyl-sn-glycero-3-phosphoethanolamine (SOPE). While several non-cationic lipids are described here, additional non-cationic lipids can be used in combination with the compounds disclosed herein.

In some embodiments, the nanoparticle comprises a non-cationic lipid in a molar ratio of about 10% to about 50%. In some embodiments, the nanoparticle comprises a non-cationic lipid in a molar ratio of about 10%, about 15%, about 20%, about 25%, about 30%, about 35%, or about 40% about 45%, or about 50%. In one embodiment, the nanoparticle comprises a non-cationic lipid in a molar ratio of about 30%.

In some embodiments, the nanoparticle includes a polyethylene glycol-lipid (PEG-lipid). PEG-lipid is incorporated to form a hydrophilic outer layer and stabilize the particles. Nonlimiting examples of polyethylene glycol-lipids include PEG-modified lipids such as PEG-modified phosphatidylethanolamines, PEG-modified phosphatidic acids, PEG-modified ceramides, PEG-modified dialkylamines, PEG-modified diacylglycerols, and PEG-modified dialkylglycerols. Representative polyethylene glycol-lipids include DMG-PEG, DLPE-PEGs, DMPE-PEGs, DPPC-PEGs, and DSPE-PEGs. In one embodiment, the polyethylene glycol-lipid is 1,2-dimyristoyl-sn-glycerol, methoxypolyethylene glycol (DMG-PEG). In one embodiment, the polyethylene glycol-lipid is 1,2-dimyristoyl-sn-glycerol, methoxypolyethylene glycol-2000 (DMG-PEG2000). DMG-PEGXXXX means 1,2-dimyristoyl-sn-glycerol, methoxypolyethylene glycol-XXXX, wherein XXXX signifies the molecular weight of the polyethylene glycol moiety, e.g. DMG-PEG2000 or DMG-PEG5000.

In some embodiments, the nanoparticle comprises a polyethylene glycol-lipid in a molar ratio of about 0% to about 5%. In some embodiments, the nanoparticle comprises a polyethylene glycol-lipid in a molar ratio of about 0%, about 0.25%, about 0.5%, about 0.75%, about 1%, about 1.5%, about 2%, about 3%, about 4%, or about 5%. In one embodiment, the nanoparticle comprises a polyethylene glycol-lipid in a molar ratio of about 0.75%.

In some embodiments, the nanoparticle includes a sterol. Sterols are well known to those skilled in the art and generally refers to those compounds having a perhydrocyclopentanophenanthrene ring system and having one or more OH substituents.

Examples of sterols include, but are not limited to, cholesterol, campesterol, ergosterol, sitosterol, and the like.

In some embodiments, the sterol is selected from a cholesterol-based lipid. In some embodiments, the one or more cholesterol-based lipids are selected from cholesterol, PEGylated cholesterol, DC-Choi (N,N-dimethyl-N-ethylcarboxamidocholesterol), 1,4-bis(3-N-oleylamino-propyl)piperazine, or combinations thereof.

The sterol can be used to tune the particle permeability and fluidity base on its function in cell membranes. In one embodiment, the sterol is cholesterol.

In some embodiments, the nanoparticle comprises a sterol in a molar ratio of about 20% to about 60%. In some embodiments, the nanoparticle comprises a sterol in a molar ratio of about 20%, about 25%, about 30%, about 35%, about 40%, about 45%, about 50%, about 55%, or about 60%. In one embodiment, the nanoparticle comprises a sterol in a molar ratio of about 40%.

In one embodiment, the disclosure provides a nanoparticle comprising:
a compound of Formula A, I, II, III, IV, V, VI, VII, VIII, IX, or X;
1,2-dioleoyl-sn-glycero-3-phosphoethanolamine (DOPE);
1,2-dimyristoyl-sn-glycerol, methoxypolyethylene glycol (DMG-PEG₂₀₀₀); and cholesterol.

In one embodiment, the disclosure provides a nanoparticle comprising:
a compound of Formula A, I, II, III, IV, V, VI, VII, VIII, IX, or X;
1-palmitoyl-2-oleoyl-sn-glycero-3-phosphoethanolamine (POPE);
1,2-dimyristoyl-sn-glycerol, methoxypolyethylene glycol (DMG-PEG₂₀₀₀); and cholesterol.

In one embodiment, the disclosure provides a nanoparticle comprising:
a compound of Formula A, I, II, III, IV, V, VI, VII, VIII, IX, or X;
1,2-distearoyl-sn-glycero-3-phosphocholine (DSPC);
1,2-dimyristoyl-sn-glycerol, methoxypolyethylene glycol (DMG-PEG₂₀₀₀); and cholesterol.

In some embodiments, the nanoparticle comprises a ratio of a compound of Formula A, I, II, III, IV, V, VI, VII, VIII, IX, or X/a non-cationic lipid/a sterol/a polyethylene glycol-lipid of 20:30:40:0.75. In some embodiments, the nanoparticle comprises a ratio of a compound of Formula A, I, II, III, IV, V, VI, VII, VIII, IX, or X/DOPE/cholesterol/PEG-lipid of 20:30:40:0.75.

In some embodiments, the nanoparticle comprises a ratio of a compound of Formula A, I, II, III, IV, V, VI, VII, VIII, IX, or X/a non-cationic lipid/a sterol/a polyethylene glycol-lipid of 40:30:40:0.75. In some embodiments, the nanoparticle comprises a ratio of a compound of Formula A, I, II, III, IV, V, VI, VII, VIII, IX, or X/DOPE/cholesterol/PEG-lipid of 40:30:40:0.75.

In some embodiments, the nanoparticle comprises a ratio of a compound of Formula A, I, II, III, IV, V, VI, VII, VIII, IX, or X/a non-cationic lipid/a sterol/a polyethylene glycol-lipid of 60:30:40:0.75. In some embodiments, the nanoparticle comprises a ratio of a compound of Formula A, I, II, III, IV, V, VI, VII, VIII, IX, or X/DOPE/cholesterol/PEG-lipid of 60:30:40:0.75.

In one embodiment, the nanoparticle further comprises an agent. In one embodiment, the nanoparticle further comprises a therapeutic agent. In one embodiment, the nanoparticle further comprises a diagnostic agent.

The agent delivered into cells can be a polynucleotide. Polynucleotides or oligonucleotides that can be introduced according to the methods herein include DNA, cDNA, and RNA sequences of all types. For example, the polynucleotide can be double stranded DNA, single-stranded DNA, complexed DNA, encapsulated DNA, naked RNA, encapsulated RNA, messenger RNA (mRNA), tRNA, short interfering RNA (siRNA), double stranded RNA (dsRNA), micro-RNA (miRNA), antisense RNA (asRNA) and combinations thereof. The polynucleotides can also be DNA constructs, such as expression vectors, expression vectors encoding a desired gene product (e.g., a gene product homologous or heterologous to the subject into which it is to be introduced), and the like. In one embodiment, the agent is an mRNA. In one embodiment, the agent is a DNA.

Compositions

Compositions, as described herein, comprising an active compound and an excipient of some sort may be useful in a variety of medical and non-medical applications. For example, pharmaceutical compositions comprising an active compound and an excipient may be useful in the delivery of an effective amount of an agent to a subject in need thereof. Nutraceutical compositions comprising an active compound and an excipient may be useful in the delivery of an effective amount of a nutraceutical, e.g., a dietary supplement, to a subject in need thereof. Cosmetic compositions comprising an active compound and an excipient may be formulated as a cream, ointment, balm, paste, film, or liquid, etc., and may be useful in the application of make-up, hair products, and materials useful for personal hygiene, etc. Compositions comprising an active compound and an excipient may be useful for non-medical applications, e.g., such as an emulsion or emulsifier, useful, for example, as a food component, for extinguishing fires, for disinfecting surfaces, for oil cleanup, etc.

In certain embodiments, the composition further comprises an agent, as described herein. For example, in certain embodiments, the agent is a small molecule, organometallic compound, nucleic acid, protein, peptide, polynucleotide, metal, targeting agent, an isotopically labeled chemical compound, drug, vaccine, immunological agent, or an agent useful in bioprocessing. In certain embodiments, the agent is a polynucleotide. In certain embodiments, the polynucleotide is DNA or RNA. In certain embodiments, the RNA is mRNA, RNAi, dsRNA, siRNA, shRNA, miRNA, or antisense RNA. In certain embodiments, the polynucleotide and the one or more active compounds are not covalently attached.

In one aspect, the disclosure provides a composition comprising:
a compound of Formula A, I, II, III, IV, V, VI, VII, VIII, IX, or X; and an agent.

In one aspect, the disclosure provides a composition comprising:
a nanoparticle, comprising a compound of Formula A, I, II, III, IV, V, VI, VII, VIII, IX, or X; and
an agent.

In another aspect, disclosed herein is a composition comprising:
a nanoparticle, comprising a compound of Formula A, I, II, III, IV, V, VI, VII, VIII, IX, or X; and an agent,
wherein the agent comprises an mRNA encoding at least one antigenic polypeptide or an immunogenic fragment thereof capable of inducing an immune response to the antigenic polypeptide.

In some embodiments, the mRNA encoding at least one antigenic polypeptide or an immunogenic fragment thereof capable of inducing an immune response to the antigenic polypeptide is encapsulated by the nanoparticle.

In some aspects, disclosed herein is a pharmaceutical composition comprising a pharmaceutically acceptable carrier and a nanoparticle comprising an mRNA at least one antigenic polypeptide or an immunogenic fragment thereof capable of inducing an immune response to the antigenic polypeptide.

Agents

Agents to be delivered by the compounds, compositions, and systems described herein may be therapeutic, diagnostic, or prophylactic agents. Any chemical compound to be administered to a subject may be delivered using the particles or nanoparticles described herein. The agent may be an organic molecule (e.g., a therapeutic agent, a drug), inorganic molecule, nucleic acid, protein, amino acid, peptide, polypeptide, polynucleotide, targeting agent, isotopically labeled organic or inorganic molecule, vaccine, immunological agent, etc.

In certain embodiments, the agents are organic molecules with pharmaceutical activity, e.g., a drug. In certain embodiments, the drug is an antibiotic, anti-viral agent, anesthetic, steroidal agent, anti-inflammatory agent, anti-neoplastic agent, anti-cancer agent, antigen, vaccine, antibody, decongestant, antihypertensive, sedative, birth control agent, progestational agent, anti-cholinergic, analgesic, anti-depressant, anti-psychotic, f3-adrenergic blocking agent, diuretic, cardiovascular active agent, vasoactive agent, non-steroidal anti-inflammatory agent, nutritional agent, etc.

In certain embodiments of the present disclosure, the agent to be delivered may be a mixture of agents.

Diagnostic agents include gases; metals; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

Therapeutic and prophylactic agents include, but are not limited to, antibiotics, nutritional supplements, and vaccines. Vaccines may comprise isolated proteins or peptides, inactivated organisms and viruses, dead organisms and viruses, genetically altered organisms or viruses, cell extracts, and RNA encoding at least one antigenic polypeptide or an immunogenic fragment thereof (e.g., an immunogenic fragment capable of inducing an immune response to the antigenic polypeptide). Therapeutic and prophylactic agents may be combined with interleukins, interferon, cytokines, and adjuvants such as cholera toxin, alum, Freund's adjuvant, etc. Prophylactic agents include antigens of such bacterial organisms as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In some aspects, the agent is a ribonucleic acid (RNA) (e.g., mRNA) polynucleotide having an open reading frame encoding at least one (e.g., at least 2, 3, 4 or 5) antigenic polypeptide or an immunogenic fragment thereof (e.g., an immunogenic fragment capable of inducing an immune response to the antigenic polypeptide).

In some embodiments, the RNA (e.g., mRNA) maybe used to induce a balanced immune response against respiratory viruses. The term “respiratory viruses” refers herein to viruses causing respiratory diseases. For example, negative-sense, single-stranded RNA virus of the family Paramyxoviridae such as human Metapneumovirus (hMPV), human parainfluenza viruses (hPIV) types 1, 2, and 3 (hPIV1, hPIV2 and hPIV3, respectively), RSV, and Measles virus (MeV). Another example of respiratory viruses are coronaviruses. Coronaviruses are enveloped viruses with a positive-sense single-stranded RNA genome and with a nucleocapsid of helical symmetry. Coronaviruses are species of virus belonging to the subfamily Coronavirinae in the family Coronaviridae, in the order Nidovirales.

Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang20l3), a merbecovirus (e.g., Hedgehog coronavirus 1, Middle East respiratory syndrome-related coronavirus (MERS-CoV), Pipistrellus bat coronavirus HKU5, Tylonycteris bat coronavirus HKU4), a nobecovirus (e.g., Rousettus bat coronavirus GCCDC1, Rousettus bat coronavirus HKU9), a sarbecovirus (e.g., severe acute respiratory syndrome coronavirus (SARS-CoV), severe acute respiratory syndrome coronavirus 2 (SARS-CoV-2).

Representative examples of gammacoronaviruses include, but are not limited to, a cegacovirus (e.g., Beluga whale coronavirus SQ1) and an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), and a moordecovirus (e.g., Common moorhen coronavirus HKU21).

In some embodiments, the coronavirus is a human coronavirus. Representative examples of human coronaviruses include, but are not limited to, human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-CoV), severe acute respiratory syndrome coronavirus 2 (SARS-CoV-2), and Middle East respiratory syndrome-related coronavirus (MERS-CoV).

In some embodiments, the RNA (e.g., mRNA) polynucleotide has an open reading frame encoding at least one (e.g., at least 2, 3, 4 or 5) hMPV, PIV, RSV, MeV, or a BetaCoV (e.g., MERS-CoV, SARS-CoV, SARS-CoV2, HCoV-OC43, HCoV-229E, HCoV-NL63, HCoV-NL, HCoV—NH, HCoV-HKU1) antigenic polypeptide, or any combination of two or more of the antigenic polypeptides. Herein, use of the term “antigenic polypeptide” encompasses immunogenic fragments of the antigenic polypeptide (an immunogenic fragment that induces (or is capable of inducing) an immune response to hMPV, PIV, RSV, MeV, or a BetaCoV), unless otherwise stated.

In some embodiments, the agent is an RNA (e.g., mRNA) vaccine that can induce a balanced immune response against hMPV, PIV, RSV, MeV, and/or BetaCoV (e.g., MERS-CoV, SARS-CoV, SARS-CoV2, HCoV-OC43, HCoV-229E, HCoV-NL63, HCoV-NL, HCoV—NH and/or HCoV-HKU1), or any combination of two or more of the foregoing viruses, comprising both cellular and humoral immunity, without risking the possibility of insertional mutagenesis, for example.

In some embodiments, the agent to be delivered is used in gene therapy. In some embodiments, the agent to be delivered is used in gene editing. In some embodiments, the agent to be delivered is used for CRISPR mediated gene editing. In some embodiments, the agent to be delivered is a Cas9 mRNA. In some embodiments, the agent to be delivered is a Cpf1 mRNA. In some embodiments, the agent to be delivered is a guide RNA.

Methods

In one aspect, provided herein is a method for the delivery of an agent (for example, a polynucleotide) into a cell comprising;
introducing into the cell a composition comprising;
a nanoparticle, comprising;
   a compound of Formula A, I, II, III, IV, V, VI, VII, VIII, IX, or X;
   a non-cationic lipid;
   a polyethylene glycol-lipid;
   a sterol; and
an agent.

In one aspect, disclosed herein is a method for the delivery of an agent into a cell comprising;
introducing into the cell a composition comprising;
a nanoparticle comprising;
   a compound of Formula A:

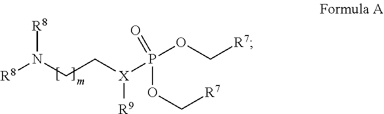

or a salt thereof,
wherein:
each R⁷ is independently selected from alkyl, alkenyl, alkynyl, ester, alkylester, alkylketal, alkylcarbonate, or alkylcarbamate;
each R⁸ is independently selected from alkyl, alkenyl, alkynyl, ester, alkylester, or alkylalcohol; R⁹ is selected from hydrogen, alkyl, alkenyl, alkynyl, ester, alkylester, or

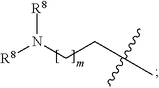

X is selected from O or N; and
m is an integer from 0 to 10; and
   an agent.

In one aspect, disclosed herein is a method for the delivery of an agent into a cell comprising;
introducing into the cell a composition comprising;
   a nanoparticle comprising;
      a compound of Formula I, II, III, IV, V, VI, VII, VIII, IX, or X;

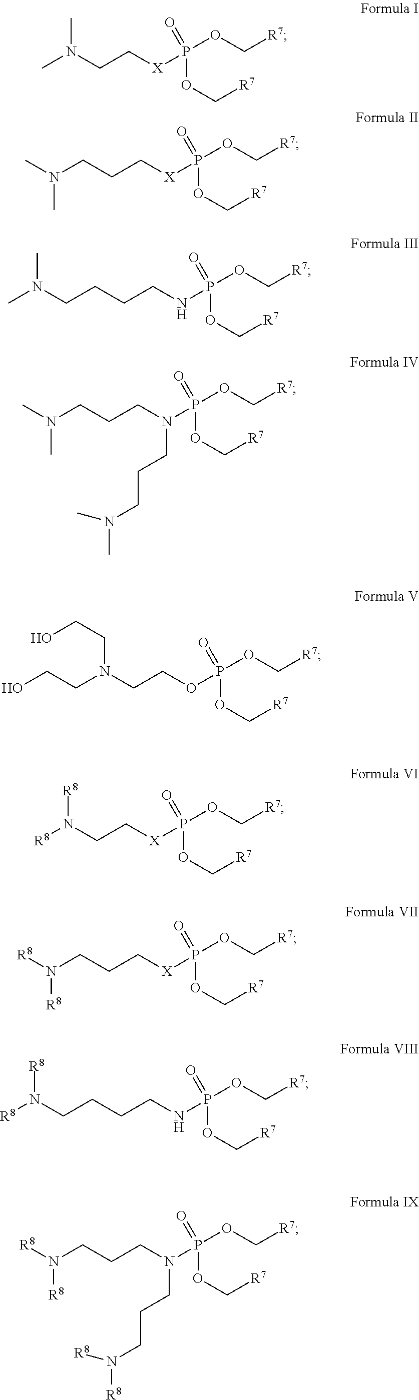

or a salt thereof,
wherein:
each R⁷ is independently selected from alkyl, alkenyl, alkynyl, ester, alkylester, alkylketal, alkylcarbonate, or alkylcarbamate;
each R⁸ is independently selected from alkyl, alkenyl, alkynyl, ester, alkylester, or alkylalcohol; and
X is selected from O or N;
   a non-cationic lipid;
   a polyethylene glycol-lipid;
   a sterol; and
an agent.

In some embodiments, a nanoparticle comprising any compound as described in the Compounds section above, is used in the methods herein, for delivery of an agent into a cell.

In some embodiments, the agent is a polynucleotide. In some embodiments, the agent is an RNA. In some embodiments, the agent is an mRNA. In some embodiments, the agent is a DNA. In some embodiments, the agent is a therapeutic agent, diagnostic agent, or prophylactic agent.

In some embodiments, provided herein are methods for the delivery of polynucleotides. In some embodiments, provided herein are methods for the delivery of polynucleotides (for example, mRNA) to correct a mutation in a genome. For example, mRNAs can be delivered to correct mutations that cause hemophilia (due to mutations in the genes encoding Factor VIII (F8; hemophilia A) or Factor IX (F9; hemoglobin B). In some embodiments, provided herein are methods for the delivery of polynucleotides. In some embodiments, provided herein are methods for the delivery of polynucleotides (for example, mRNA) to provide expression of the mRNA (and translation to produce a protein) in a cell. In some embodiments, provided herein are methods for the delivery of polynucleotides (for example, mRNA) to induce an immune response in a subject. In some embodiments, the RNA (e.g., mRNA) polynucleotide having an open reading frame encoding at least one (e.g., at least 2, 3, 4 or 5) hMPV, PIV, RSV, MeV, and/or a BetaCoV (e.g., MERS-CoV, SARS-CoV, SARS-CoV2, HCoV-OC43, HCoV-229E, HCoV-NL63, HCoV-NL, HCoV—NH, HCoV-HKU1) antigenic polypeptide, or any combination of two or more of the antigenic polypeptides.

In some embodiments, the subject is a mammal. In some embodiments, the mammal is a human.

### Examples

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Bioinspired Lipids

Efficient delivery of mRNA is a key step and challenge for the application of mRNA therapeutics. Despite promising data from ongoing clinical trials, the clinical use of mRNA requires the discovery and development of more efficient delivery systems.

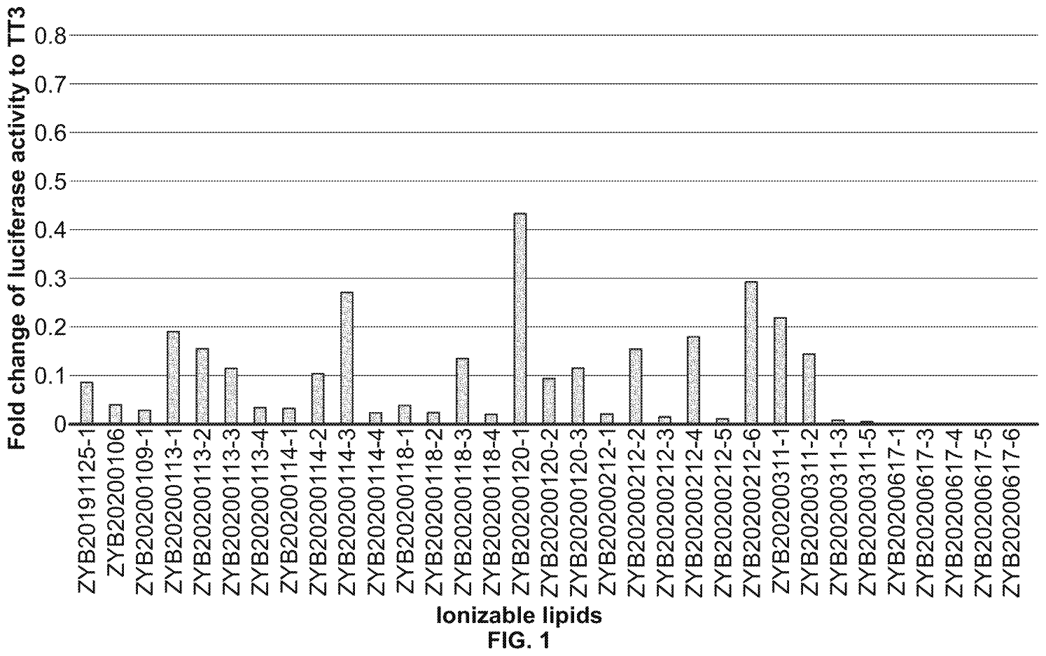

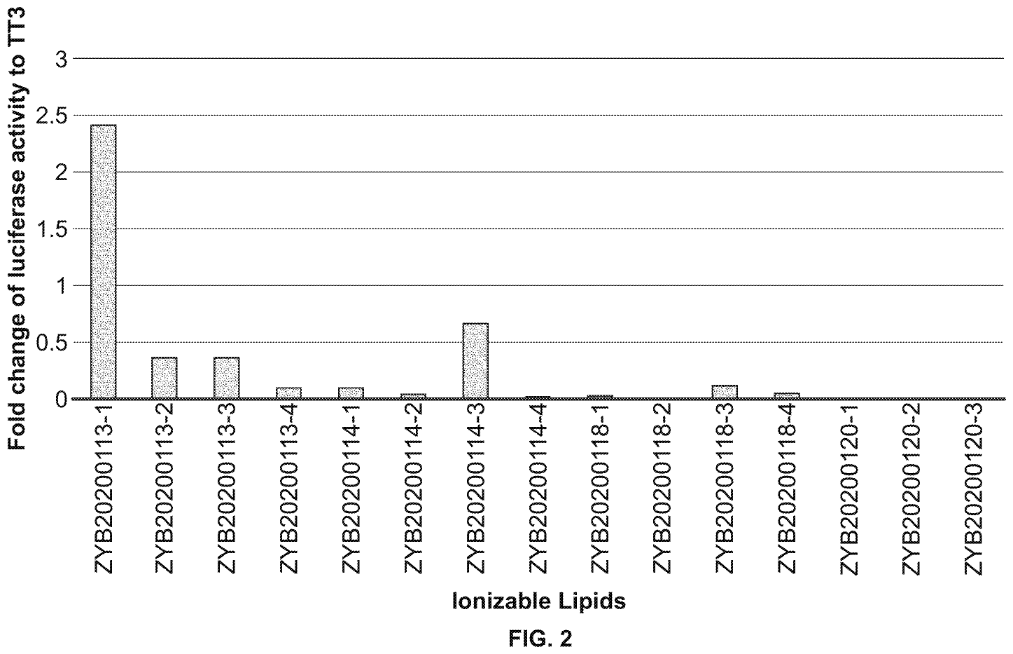

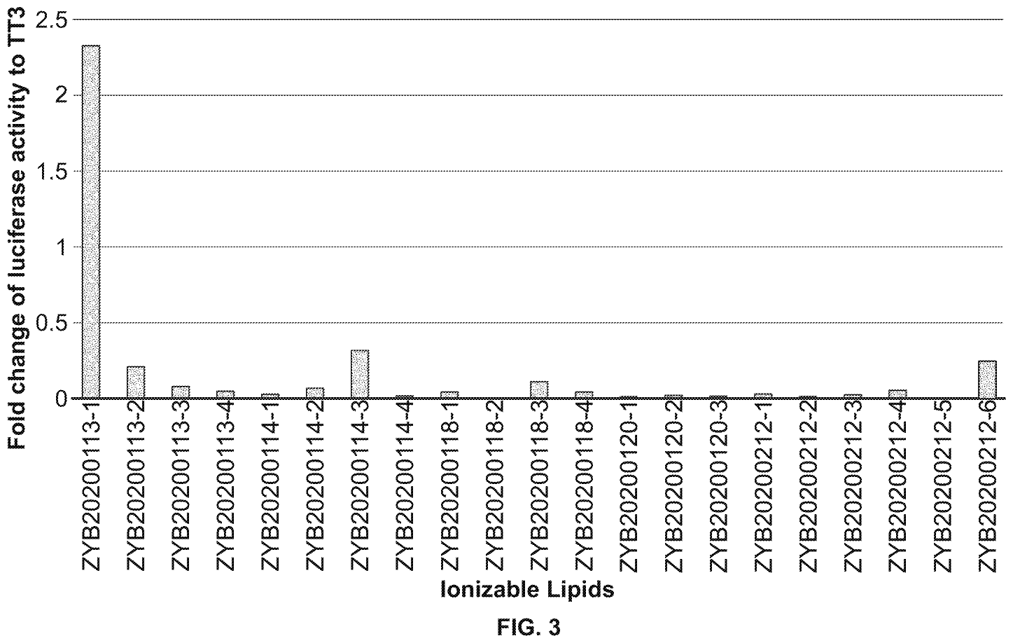

Phosphates and phosporamide lipid-like compounds First, forty ionizable lipids were synthesized. Then, these ionizable lipids were formulated with DOPE/cholesterol/PEG-lipid at three different ratios (20:30:40:0.75, 40:30:40:0.75, and 60:30:40:0.75) to prepare lipid nanoparticles (LNPs) and screened in Hep3b cells using mRNA-encoding firefly luciferase. ZYB20200113-1 was more effective for mRNA delivery than other ionizable lipids (FIGS. 1-3).

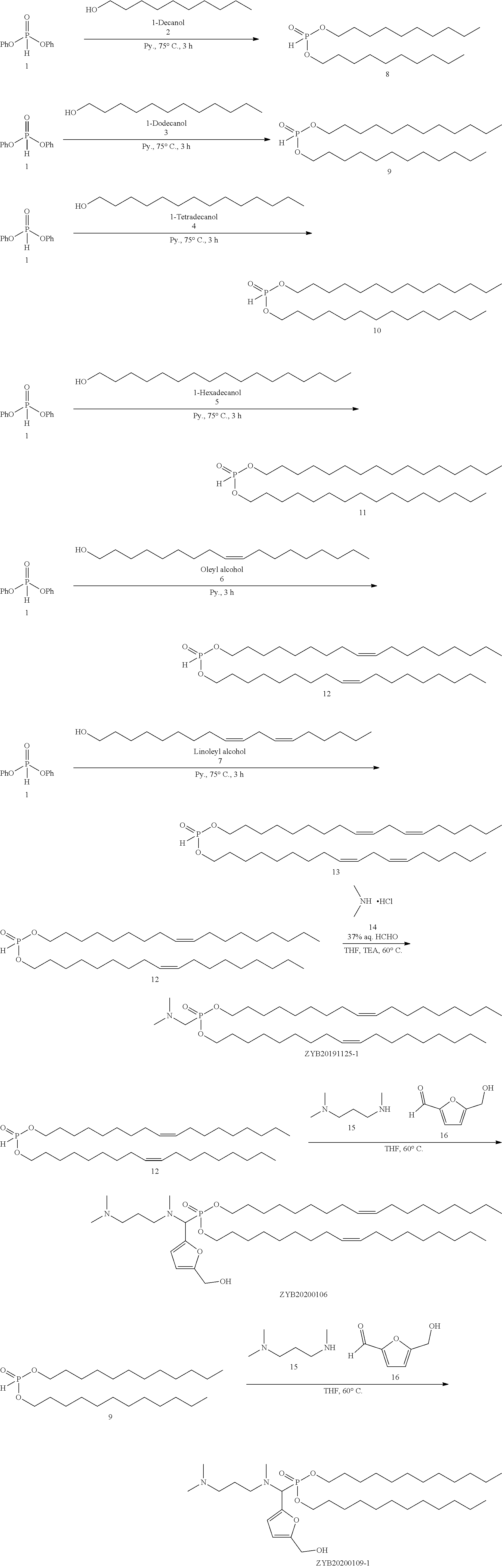

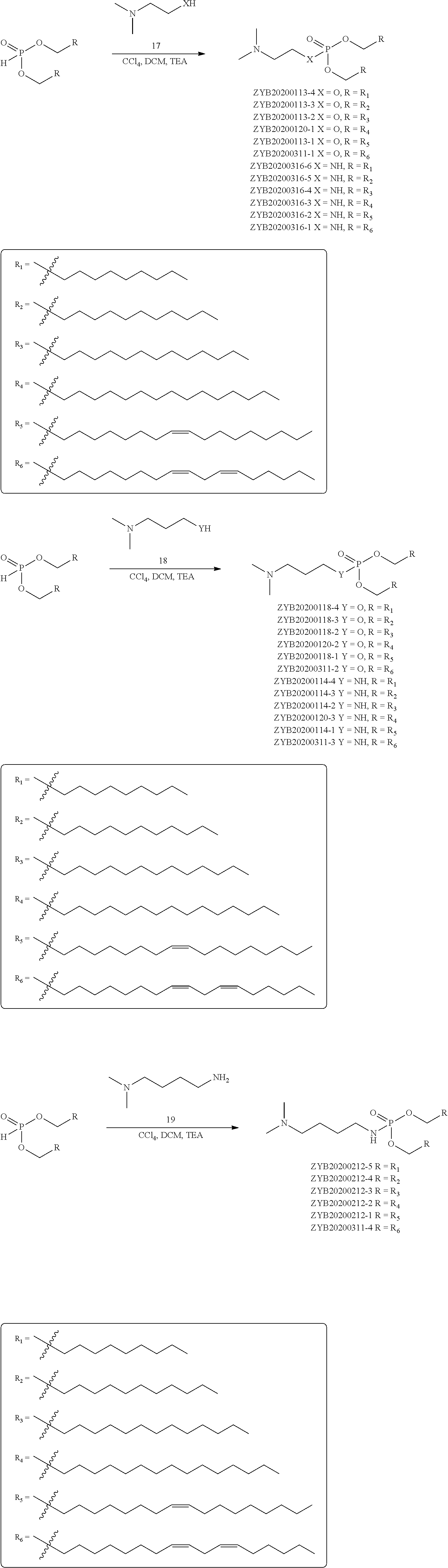

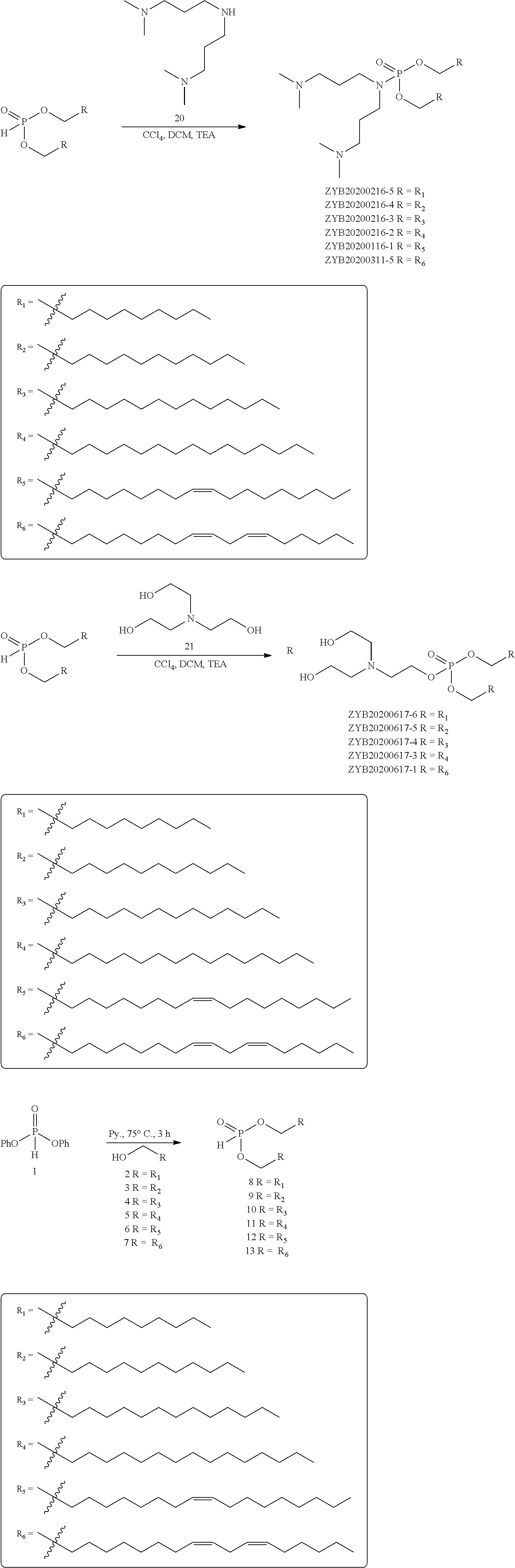

### General Procedure for the Synthesis of Dialkyl-H-Phosphonates

To a solution of diphenyl phosphonate 1 (0.7 g, 3.0 mmol) in 3.0 mL of pyridine was added alcohol (6.15 mmol). The resulting solution was then allowed to warmed to 75° C. and stirred for 3 h. Pyridine was removed under reduced pressure, the residue was diluted with 100 mL of DCM and washed with 10 mL of 1 N aqueous NaOH solution and 10 mL of water. The organic phase was dried over anhydrous Na₂SO₄, filtered, and the solvent was removed under reduced pressure. The residue was purified by silica gel chromatography (0%-10% Ethyl acetate in Hexane) to give the desired products.

### General Procedure for the Synthesis of the Amino Phosphate and Phosphamide Lipids

To a flame-dried flask containing Dialkyl-H-Phosphonates (0.35 mmol) and carbon tetrachloride (2.0 mL) was added dropwise a solution of trimethylamine (194.6 μL, 1.4 mmol), DMAP (4.3 mg, 0.035 mmol), and amino alcohols or amines (2.0 mmol) in 1.0 mL of dry DCM under vigorous stirring at RT. The reaction mixture was stirred for 1 h, diluted with 50 mL of DCM, and washed three times with 50 mL of brine. The organic phase was isolated, dried over anhydrous Na₂SO₄, filtered, and the solvent was removed in vacuo. The residue was purified via silica gel chromatography (0%-20% [mixture of 3% NH₄OH, 22% MeOH in dichloromethane] in dichloromethane) to give desired products.

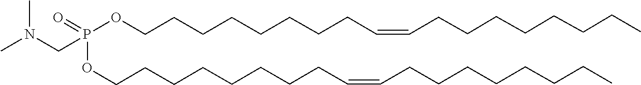

ZYB20191125-1. ¹H NMR (300 MHz, CDCl₃) δ 5.45-5.27 (m, 4H), 4.08 (qd, J=6.8, 1.5 Hz, 4H), 2.77 (d, J=11.5 Hz, 2H), 2.42 (s, 6H), 2.03 (q, J=6.7, 5.6 Hz, 8H), 1.69 (p, J=6.8 Hz, 4H), 1.44-1.20 (m, 44H), 0.90 (t, J=6.9 Hz, 6H). ³¹P NMR (121 MHz, CDCl₃) δ 25.07. MS (m z): [M+H]⁺ calcd. for C₃₉H₇₉NO₃P⁺, 640.5792; found: 640.5797.

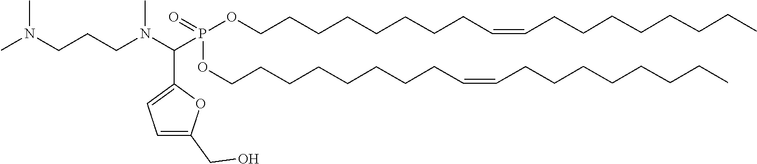

ZYB20200106. ¹H NMR (300 MHz, CDCl₃) δ 6.43 (dd, J=3.2, 1.6 Hz, 1H), 6.25 (d, J=3.2 Hz, 1H), 5.45-5.26 (m, 4H), 4.56 (s, 2H), 4.23-4.06 (m, 3H), 3.97 (dq, J=10.5, 7.0 Hz, 1H), 3.85 (dt, J=10.0, 7.1 Hz, 1H), 2.71 (dd, J=13.0, 7.0 Hz, 1H), 2.49 (t, J=6.8 Hz, 1H), 2.43-2.33 (m, 5H), 2.27 (s, 6H), 2.01 (d, J=6.4 Hz, 8H), 1.66 (hept, J=7.0 Hz, 4H), 1.51 (t, J=6.6 Hz, 2H), 1.39-1.23 (m, 44H), 0.88 (t, J=6.6 Hz, 3H). ³¹P NMR (121 MHz, CDCl₃) δ 21.04.

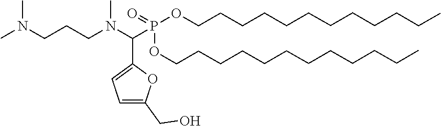

ZYB20200109-1. ¹H NMR (300 MHz, CDCl₃) δ 6.43 (dd, J=3.2, 1.6 Hz, 1H), 6.24 (d, J=3.2 Hz, 1H), 4.56 (s, 2H), 4.20-4.07 (m, 3H), 4.02-3.91 (m, 1H), 3.91-3.80 (m, 1H), 2.69 (dt, J=13.8, 7.2 Hz, 1H), 2.46 (dt, J=13.4, 7.1 Hz, 1H), 2.38 (s, 3H), 2.35-2.30 (m, 2H), 2.24 (s, 3H), 1.74-1.56 (m, 4H), 1.51 (t, J=6.6 Hz, 2H), 1.32-1.22 (m, 36H), 0.88 (t, J=6.6 Hz, 6H). ³¹P NMR (121 MHz, CDCl₃) δ 21.28.

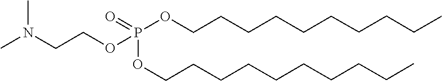

ZYB20200113-4. ¹H NMR (300 MHz, CDCl₃) δ 4.13 (dt, J=7.6, 6.0 Hz, 2H), 4.03 (q, J=6.9 Hz, 4H), 2.63 (t, J=6.0 Hz, 2H), 2.30 (s, 6H), 1.67 (p, J=6.6 Hz, 4H), 1.38-1.25 (m, 28H), 0.88 (t, J=6.6 Hz, 6H). ³¹P NMR (121 MHz, CDCl₃) δ-0.24.

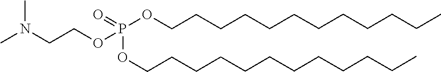

ZYB20200113-3. ¹H NMR (300 MHz, CDCl₃) δ 4.13 (dt, J=7.5, 6.0 Hz, 2H), 4.03 (q, J=6.6 Hz, 4H), 2.63 (t, J=6.0 Hz, 2H), 2.31 (s, 6H), 1.67 (p, J=6.6 Hz, 4H), 1.43-1.18 (m, 36H), 0.87 (t, J=6.6 Hz, 6H). ³¹P NMR (121 MHz, CDCl₃) δ-0.40.

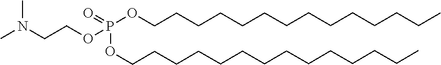

ZYB20200113-2. ¹H NMR (300 MHz, CDCl₃) δ 4.13 (dt, J=7.5, 6.0 Hz, 2H), 4.03 (q, J=6.6 Hz, 4H), 2.63 (t, J=6.0 Hz, 2H), 2.31 (s, 6H), 1.67 (p, J=6.6 Hz, 4H), 1.38-1.25 (m, 44H), 0.88 (t, J=6.6 Hz, 6H). ³¹P NMR (121 MHz, CDCl₃) δ-0.24.

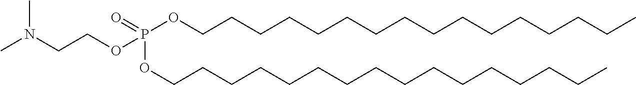 [SMILES: CCCCCCCCCCCCCCCCOP(=O)(OCCCCCCCCCCCCCCCC)OCCN(C)C]

ZYB20200120-1. ¹H NMR (300 MHz, CDCl₃) δ 4.13 (q, J=6.3 Hz, 2H), 4.03 (q, J=6.9 Hz, 4H), 2.62 (t, J=6.0 Hz, 2H), 2.30 (s, 6H), 1.68 (q, J=6.9 Hz, 4H), 1.36-1.25 (m, 52H), 0.88 (m, 6H). ³¹P NMR (121 MHz, CDCl₃) δ-0.23.

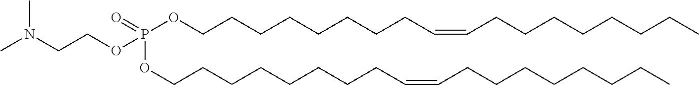 [SMILES: CCCCCCCC/C=C\CCCCCCCCOP(=O)(OCCCCCCCC/C=C\CCCCCCCC)OCCN(C)C]

ZYB20200113-1. ¹H NMR (300 MHz, CDCl₃) δ 5.47-5.26 (m, 4H), 4.13 (dt, J=7.5, 6.0 Hz, 2H), 4.03 (q, J=6.9 Hz, 4H), 2.63 (t, J=6.0 Hz, 2H), 2.30 (s, 6H), 2.00 (t, J=6.3 Hz, 8H), 1.68 (q, J=6.9 Hz, 4H), 1.38-1.25 (m, 44H), 0.88 (t, J=6.6 Hz, 6H). ³¹P NMR (121 MHz, CDCl₃) δ-0.24.

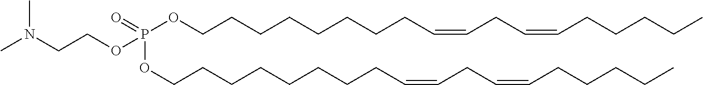 [SMILES: CCCCC/C=C\C/C=C\CCCCCCCCOP(=O)(OCCCCCCCC/C=C\C/C=C\CCCCC)OCCN(C)C]

ZYB20200311-1. ¹H NMR (300 MHz, CDCl₃) δ 5.50-5.26 (m, 8H), 4.12 (dt, J=7.4, 6.0 Hz, 2H), 4.03 (q, J=6.7 Hz, 4H), 2.77 (t, J=6.0 Hz, 4H), 2.61 (t, J=6.0 Hz, 2H), 2.29 (s, 6H), 2.05 (q, J=6.6 Hz, 8H), 1.67 (t, J=7.2 Hz, 4H), 1.41-1.22 (m, 32H), 0.89 (t, J=6.6 Hz, 6H). ³¹P NMR (121 MHz, CDCl₃) δ-0.24. MS (m z): [M+H]⁺ calcd. for C₄₀H₇₇NO₄P⁺, 666.5585; found: 666.5594.

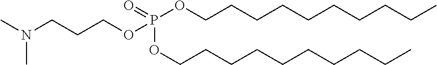 [SMILES: CCCCCCCCCCOP(=O)(OCCCCCCCCCC)OCCCN(C)C]

ZYB20200118-4. ¹H NMR (300 MHz, CDCl₃) δ 4.12-4.05 (m, 2H), 4.05-3.97 (m, 4H), 2.39 (t, J=7.2 Hz, 2H), 2.24 (s, 6H), 1.85 (p, J=6.6 Hz, 2H), 1.68 (q, J=6.9 Hz, 4H), 1.38-1.25 (m, 28H), 0.88 (t, J=6.6 Hz, 6H). ³¹P NMR (121 MHz, CDCl₃) δ-0.24.

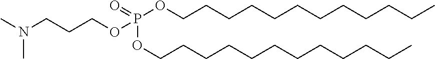 [SMILES: CCCCCCCCCCCCOP(=O)(OCCCCCCCCCCCC)OCCCN(C)C]

ZYB20200118-3. ¹H NMR (300 MHz, Chloroform-d) δ 4.15-4.06 (m, 2H), 4.02 (q, J=6.9 Hz, 4H), 2.39 (t, J=7.2 Hz, 2H), 2.24 (d, J=1.5 Hz, 6H), 1.86 (p, J=6.6 Hz, 2H), 1.67 (p, J=6.6 Hz, 4H), 1.36-1.25 (m, 36H), 0.88 (t, J=7.2 Hz, 6H). ³¹P NMR (121 MHz, CDCl₃) δ-0.39.

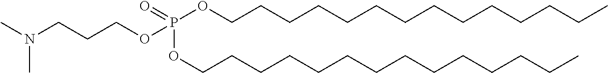 [SMILES: CCCCCCCCCCCCCCOP(=O)(OCCCCCCCCCCCCCC)OCCCN(C)C]

ZYB20200118-2. ¹H NMR (300 MHz, Chloroform-d) δ 4.14-4.05 (m, 2H), 4.05-3.96 (m, 4H), 2.44-2.33 (m, 2H), 2.24 (d, J=1.2 Hz, 6H), 1.86 (p, J=6.6 Hz, 2H), 1.68 (q, J=6.9 Hz, 4H), 1.38-1.25 (m, 44H), 0.88 (t, J=6.6 Hz, 6H). ³¹P NMR (121 MHz, CDCl₃) δ-0.25.

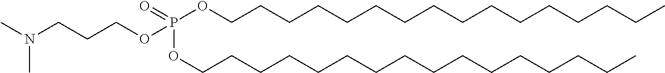 [SMILES: CCCCCCCCCCCCCCCCOP(=O)(OCCCCCCCCCCCCCCCC)OCCCN(C)C]

ZYB20200120-2. H NMR (300 MHz, Chloroform-d) δ 4.14-4.05 (m, 2H), 4.05-3.95 (m, 4H), 2.48-2.33 (m, 2H), 2.23 (s, 6H), 1.93-1.78 (m, 2H), 1.68 (q, J=6.9 Hz, 4H), 1.38-1.25 (m, 52H), 0.87 (t, J=6.6 Hz, 6H). ³¹P NMR (121 MHz, CDCl₃) δ-0.39.

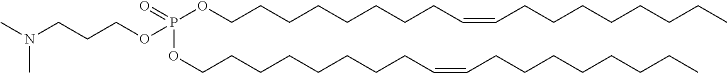 [SMILES: CCCCCCCC/C=C\CCCCCCCCOP(=O)(OCCCCCCCC/C=C\CCCCCCCC)OCCCN(C)C]

ZYB20200118-1. H NMR (300 MHz, Chloroform-d) δ 5.44-5.25 (m, 4H), 4.13-4.05 (m, 2H), 4.05-3.98 (m, 4H), 2.40 (t, J=7.5 Hz, 2H), 2.25 (s, 6H), 2.00 (t, J=6.3 Hz, 7H), 1.87 (p, J=6.9 Hz, 2H), 1.68 (q, J=6.9 Hz, 4H), 1.44-1.18 (m, 44H), 0.88 (t, J=6.6 Hz, 6H). ³¹P NMR (121 MHz, CDCl₃) δ-0.20.

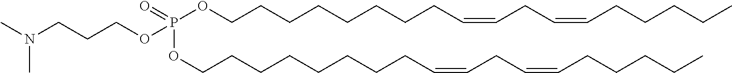 [SMILES: CCCCC/C=C\C/C=C\CCCCCCCCOP(=O)(OCCCCCCCC/C=C\C/C=C\CCCCC)OCCCN(C)C]

ZYB20200311-2. ^{l}H NMR (300 MHz, Chloroform-d) δ 5.45-5.26 (m, 8H), 4.15-4.05 (m, 2H), 4.05-3.96 (m, 4H), 2.77 (t, J=6.0 Hz, 4H), 2.36 (t, J=7.2 Hz, 2H), 2.22 (s, 6H), 2.05 (q, J=6.6 Hz, 8H), 1.84 (p, J=6.9 Hz, 2H), 1.73-1.63 (m, 4H), 1.39-1.22 (m, 32H), 0.89 ((t, J=6.6 Hz, 6H). ³¹P NMR (121 MHz, CDCl₃) δ-1.7. MS (m z): [M+H]⁺ calcd. for C₄₁H₇₉NO₄P⁺, 680.5741; found: 680.5748.

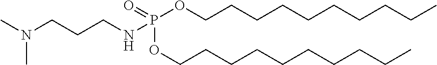 [SMILES: CCCCCCCCCCOP(=O)(NCCCN(C)C)OCCCCCCCCCC]

ZYB20200114-4. ^{l}H NMR (300 MHz, Chloroform-d) δ 3.96 (qd, J=6.6, 3.0 Hz, 4H), 3.51 (dt, J=12.3, 6.3 Hz, 1H), 2.98 (dq, J=9.6, 6.3 Hz, 2H), 2.37 (t, J=6.6 Hz, 2H), 2.22 (s, 6H), 1.65 (ddt, J=11.5, 6.9, 4.2 Hz, 6H), 1.31-1.24 (m, 28H), 0.87 (t, J=6.6 Hz, 6H). ³¹P NMR (121 MHz, CDCl₃) δ 9.61.

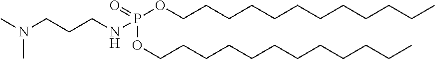 [SMILES: CCCCCCCCCCCCOP(=O)(NCCCN(C)C)OCCCCCCCCCCCC]

ZYB20200114-3. ¹H NMR (300 MHz, Chloroform-d) δ 3.98 (qd, J=6.6, 3.0 Hz, 4H), 3.53 (dt, J=12.0, 6.6 Hz, 1H), 3.00 (dq, J=9.6, 6.6 Hz, 2H), 2.38 (t, J=6.6 Hz, 2H), 2.24 (s, 6H), 1.67 (ddt, J=9.6, 6.6, 4.8 Hz, 6H), 1.46-1.18 (m, 36H), 0.90 (t, J=6.6 Hz, 6H). ³¹P NMR (121 MHz, CDCl₃) δ 9.58.

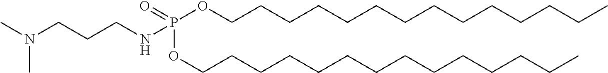 [SMILES: CCCCCCCCCCCCCCOP(=O)(NCCCN(C)C)OCCCCCCCCCCCCCC]

ZYB20200114-2. ¹H NMR (300 MHz, Chloroform-d) δ 3.96 (qd, J=6.6, 3.3 Hz, 4H), 3.51 (dt, J=12.0, 6.0 Hz, 1H), 2.98 (dq, J=9.6, 6.6 Hz, 2H), 2.37 (dd, J=7.5, 5.7 Hz, 2H), 2.22 (t, J=1.2 Hz, 6H), 1.71-1.58 (m, 6H), 1.38-1.25 (m, 44H), 0.88 (q, J=6.6, Hz, 6H). ³¹P NMR (121 MHz, CDCl₃) δ 9.58.

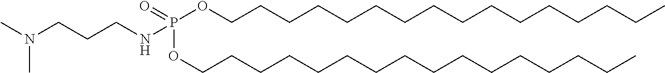 [SMILES: CCCCCCCCCCCCCCCCOP(=O)(NCCCN(C)C)OCCCCCCCCCCCCCCCC]

ZYB20200120-3. ¹H NMR (300 MHz, Chloroform-d) δ 3.96 (qd, J=6.6, 3.3 Hz, 4H), 2.98 (dq, J=9.6, 6.6 Hz, 2H), 2.36 (t, J=6.6 Hz, 2H), 2.21 (s, 6H), 1.64 (ddt, J=9.6, 6.6, 3.9 Hz, 6H), 1.31-1.25 (m, 52H), 0.87 (t, J=6.6 Hz, 6H). ³¹P NMR (121 MHz, CDCl₃) δ 9.69.

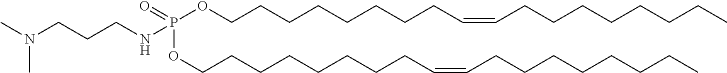 [SMILES: CCCCCCCC/C=C\CCCCCCCCOP(=O)(NCCCN(C)C)OCCCCCCCC/C=C\CCCCCCCC]

ZYB20200114-1. ¹H NMR (300 MHz, Chloroform-d) δ 5.43-5.26 (m, 2H), 3.96 (qd, J=6.6, 3.0 Hz, 2H), 2.98 (dq, J=9.6, 6.3 Hz, 1H), 2.36 (t, J=6.6 Hz, 1H), 2.22 (s, 3H), 1.99 (t, J=6.3 Hz, 4H), 1.64 (ddt, J=9.3, 6.6, 3.3 Hz, 3H), 1.32-1.25 (m, 22H), 0.87 (t, J=6.6 Hz, 3H). ³¹P NMR (121 MHz, CDCl₃) δ 9.68.

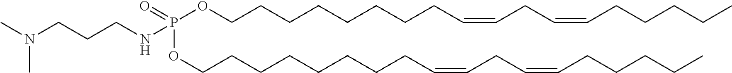 [SMILES: CCCCC/C=C\C/C=C\CCCCCCCCOP(=O)(NCCCN(C)C)OCCCCCCCC/C=C\C/C=C\CCCCC]

ZYB20200311-3. ¹H NMR (300 MHz, Chloroform-d) δ 5.46-5.25 (m, 8H), 3.96 (qd, J=6.9, 2.7 Hz, 4H), 3.53 (dt, J=12.0, 6.3 Hz, 1H), 2.98 (dq, J=9.6, 6.3 Hz, 2H), 2.77 (t, J=6.0 Hz, 4H), 2.34 (t, J=6.6 Hz, 2H), 2.20 (s, 6H), 2.04 (q, J=6.6 Hz, 8H), 1.64 (q, J=7.2 Hz, 6H), 1.41-1.22 (m, 32H), 0.89 (t, J=6.6 Hz, 6H). ³¹P NMR (121 MHz, CDCl₃) δ 9.69. MS (m z): [M+H]⁺ calcd. for C₄₁H₈₀N₂O₃P⁺, 679.5901; found: 679.5908.

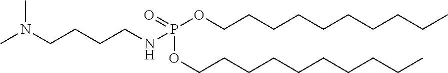 [SMILES: CCCCCCCCCCOP(=O)(NCCCCN(C)C)OCCCCCCCCCC]

ZYB20200212-5. ¹H NMR (300 MHz, Chloroform-d) δ 3.96 (qd, J=6.6, 3.9 Hz, 4H), 3.09-2.95 (m, 1H), 2.90 (dq, J=9.6, 6.3 Hz, 2H), 2.30-2.23 (m, 2H), 2.20 (s, 6H), 1.66 (q, J=6.9 Hz, 4H), 1.50 (dq, J=6.6, 3.7, 3.2 Hz, 4H), 1.37-1.24 (m, 28H), 0.87 (t, J=6.6 Hz, 6H). ³¹P NMR (121 MHz, CDCl₃) δ 9.67.

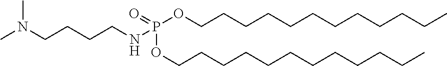 [SMILES: CCCCCCCCCCCCOP(=O)(NCCCCN(C)C)OCCCCCCCCCCCC]

ZYB20200212-4. ¹H NMR (300 MHz, Chloroform-d) δ 3.96 (qd, J=6.6, 3.9 Hz, 4H), 2.99 (dd, J=10.5, 6.6 Hz, 1H), 2.90 (dq, J=9.6, 6.3 Hz, 2H), 2.33-2.22 (m, 2H), 2.21 (s, 6H), 1.67 (q, J=6.9 Hz, 4H), 1.50 (dq, J=6.6, 3.9, 3.3 Hz, 4H), 1.35-1.25 (m, 36H), 0.87 (t, J=6.6 Hz, 6H). ³¹P NMR (121 MHz, CDCl₃) δ 9.61.

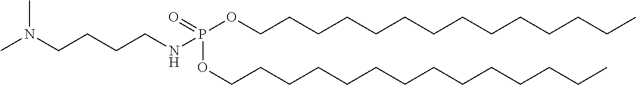 [SMILES: CCCCCCCCCCCCCCOP(=O)(NCCCCN(C)C)OCCCCCCCCCCCCCC]

ZYB20200212-3. ¹H NMR (300 MHz, Chloroform-d) δ 3.96 (qd, J=6.6, 4.0 Hz, 4H), 3.09-2.96 (m, 1H), 2.90 (dq, J=9.6, 6.3 Hz, 2H), 2.21 (s, 8H), 1.67 (q, J=6.9 Hz, 4H), 1.50 (dq, J=6.6, 3.6, 3.0 Hz, 4H), 1.35-1.25 (s, 44H), 0.87 (t, J=6.6 Hz, 6H). ³¹P NMR (121 MHz, CDCl₃) δ 9.74.

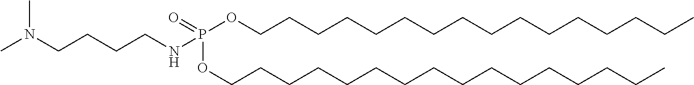 [SMILES: CCCCCCCCCCCCCCCCOP(=O)(NCCCCN(C)C)OCCCCCCCCCCCCCCCC]

ZYB20200212-2. ¹H NMR (300 MHz, Chloroform-d) δ 3.99 (qd, J=6.6, 4.2 Hz, 4H), 2.93 (dq, J=12.6, 6.3, 5.2 Hz, 3H), 2.37-2.27 (m, 2H), 2.25 (s, 6H), 1.72-1.63 (m, 4H), 1.54 (p, J=3.6 Hz, 4H), 1.38-1.27 (m, 52H), 0.90 (t, J=6.6 Hz, 6H). ³¹P NMR (121 MHz, CDCl₃) δ9.71.

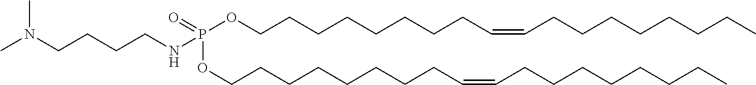 [SMILES: CCCCCCCC/C=C\CCCCCCCCOP(=O)(NCCCCN(C)C)OCCCCCCCC/C=C\CCCCCCCC]

ZYB20200212-1. ¹H NMR (300 MHz, Chloroform-d) δ 5.41-5.26 (m, 4H), 3.96 (qq, J=7.2, 3.3 Hz, 4H), 3.09-2.96 (m, 1H), 2.90 (dq, J=9.3, 6.3 Hz, 2H), 2.26 (t, J=6.6 Hz, 2H), 2.21 (s, 6H), 1.99 (t, J=6.3 Hz, 8H), 1.65 (t, J=7.2 Hz, 4H), 1.50 (dq, J=6.6, 3.7, 3.2 Hz, 4H), 1.38-1.16 (m, 44H), 0.87 (t, J=6.6 Hz, 6H). ³¹P NMR (121 MHz, CDCl₃) δ 9.76.

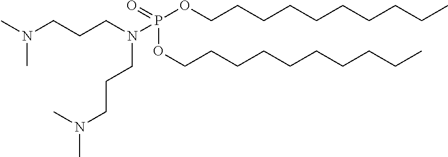 [SMILES: CCCCCCCCCCOP(=O)(OCCCCCCCCCC)N(CCCN(C)C)CCCN(C)C]

ZYB20200216-5. ¹H NMR (300 MHz, Chloroform-d) δ 4.00-3.81 (m, 4H), 3.08-2.96 (m, 4H), 2.26 (t, J=7.5 Hz, 4H), 2.22 (s, 12H), 1.68 (ddd, J=24.3, 11.4, 6.6 Hz, 8H), 1.34-1.25 (m, 28H), 0.87 (t, J=6.6 Hz, 6H). ³¹P NMR (121 MHz, CDCl₃) δ 9.60.

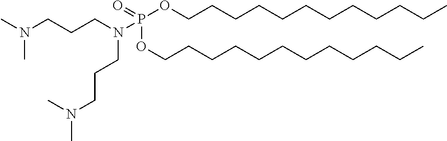 [SMILES: CCCCCCCCCCCCOP(=O)(OCCCCCCCCCCCC)N(CCCN(C)C)CCCN(C)C]

ZYB20200216-4. ¹H NMR (300 MHz, Chloroform-d) δ 4.00-3.80 (m, 4H), 3.01 (ddd, J=11.1, 8.7, 6.6 Hz, 4H), 2.24 (dd, J=8.4, 6.3 Hz, 4H), 2.20 (s, 12H), 1.67 (tt, J=14.1, 6.3 Hz, 8H), 1.29-1.25 (m, 36H), 0.87 (t, J=6.6 Hz, 6H).

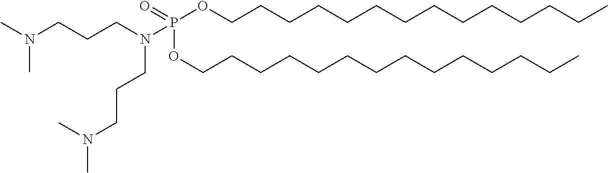 [SMILES: CCCCCCCCCCCCCCOP(=O)(OCCCCCCCCCCCCCC)N(CCCN(C)C)CCCN(C)C]

ZYB20200216-3. ¹H NMR (300 MHz, Chloroform-d) δ 4.02-3.80 (m, 4H), 3.09-2.95 (m, 4H), 2.20 (s, 16H), 1.66 (dp, J=13.8, 7.5, 6.9 Hz, 8H), 1.33-1.25 (m, 44H), 0.87 (t, J=6.6 Hz, 6H). ³¹P NMR (121 MHz, CDCl₃) δ 10.89.

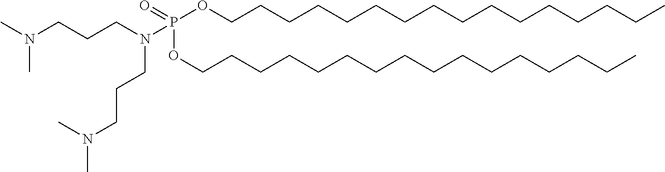 [SMILES: CCCCCCCCCCCCCCCCOP(=O)(OCCCCCCCCCCCCCCCC)N(CCCN(C)C)CCCN(C)C]

ZYB20200216-2. ¹H NMR (300 MHz, Chloroform-d) δ 4.01-3.81 (m, 4H), 3.10-2.94 (m, 4H), 2.29-2.22 (m, 4H), 2.21 (s, 12H), 1.68 (tt, J=14.1, 6.3 Hz, 8H), 1.33-1.25 (m, 52H), 0.87 (t, J=6.6 Hz, 6H).

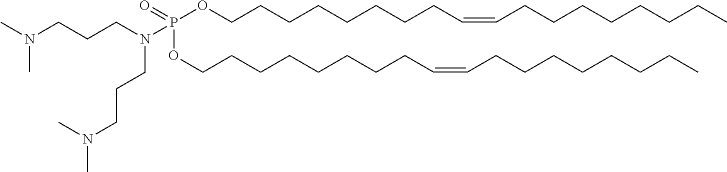 [SMILES: CCCCCCCC/C=C\CCCCCCCCOP(=O)(OCCCCCCCC/C=C\CCCCCCCC)N(CCCN(C)C)CCCN(C)C]

ZYB20200216-1. ¹H NMR (300 MHz, Chloroform-d) δ 5.34 (td, J=6.0, 5.4, 3.3 Hz, 4H), 3.98-3.81 (m, 4H), 3.10-2.95 (m, 4H), 2.51-2.13 (m, 16H), 1.99 (p, J=8.1, 7.2 Hz, 8H), 1.66 (dq, J=20.7, 7.1 Hz, 8H), 1.36-1.24 (m, 48H), 0.87 (t, J=6.6 Hz, 6H). ³¹P NMR (121 MHz, CDCl₃) δ 10.67.

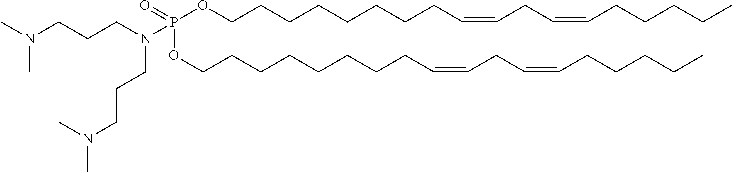 [SMILES: CCCCC/C=C\C/C=C\CCCCCCCCOP(=O)(OCCCCCCCC/C=C\C/C=C\CCCCC)N(CCCN(C)C)CCCN(C)C]

ZYB20200311-5. ¹H NMR (300 MHz, Chloroform-d) δ 5.46-5.25 (m, 8H), 4.00-3.81 (m, 4H), 3.13-2.90 (m, 4H), 2.77 (t, J=6.0 Hz, 4H), 2.24 (dd, J=8.4, 6.3 Hz, 4H), 2.21 (s, 12H), 2.04 (q, J=6.6 Hz, 8H), 1.66 (tt, J=12.9, 7.2 Hz, 8H), 1.38-1.23 (m, 32H), 0.89 (t, J=6.6 Hz, 6H). ³¹P NMR (121 MHz, CDCl₃) δ 11.01. MS (m z): [M+H]⁺ calcd. for C₄₆H₉₁N₃O₃P⁺, 764.6793; found: 764.6802.

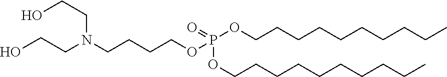 [SMILES: CCCCCCCCCCOP(=O)(OCCCCCCCCCC)OCCCCN(CCO)CCO]

ZYB20200617-6. ¹H NMR (300 MHz, Chloroform-d) δ 4.05 (qd, J=6.6, 6.0, 1.5 Hz, 6H), 3.66-3.52 (m, 4H), 2.77 (t, J=4.5 Hz, 2H), 2.70-2.53 (m, 4H), 1.65 (q, J=6.9 Hz, 4H), 1.36-1.25 (m, 28H), 0.92-0.79 (m, 6H). ³¹P NMR (121 MHz, CDCl₃) δ 0.69.

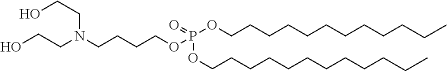 [SMILES: CCCCCCCCCCCCOP(=O)(OCCCCCCCCCCCC)OCCCCN(CCO)CCO]

ZYB20200617-5. ¹H NMR (300 MHz, Chloroform-d) δ 4.14-4.00 (m, 6H), 3.59 (t, J=4.8 Hz, 4H), 2.80-2.73 (m, 2H), 2.71-2.62 (m, 4H), 1.66 (q, J=6.9 Hz, 4H), 1.35-1.25 (m, 36H), 0.88 (t, J=6.6 Hz, 6H). ³¹P NMR (121 MHz, CDCl₃) δ 0.84.

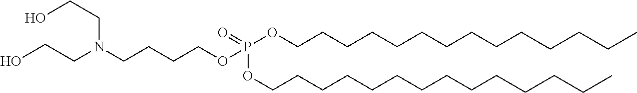 [SMILES: CCCCCCCCCCCCCCOP(=O)(OCCCCCCCCCCCCCC)OCCCCN(CCO)CCO]

ZYB20200617-4. ¹H NMR (300 MHz, Chloroform-d) δ 4.16-4.02 (m, 6H), 3.61 (t, J=4.8 Hz, 4H), 2.85-2.73 (m, 2H), 2.68 (dd, J=5.7, 3.9 Hz, 4H), 1.69 (t, J=7.1 Hz, 4H), 1.35-1.27 (m, 44H), 0.89 (t, J=6.6 Hz, 6H). ³¹P NMR (121 MHz, CDCl₃) δ 1.03.

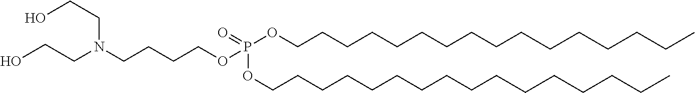 [SMILES: CCCCCCCCCCCCCCCCOP(=O)(OCCCCCCCCCCCCCCCC)OCCCCN(CCO)CCO]

ZYB20200617-3. ¹H NMR (300 MHz, Chloroform-d) δ 4.05 (q, J=6.9 Hz, 6H), 3.59 (t, J=4.8 Hz, 4H), 2.77 (t, J=4.5 Hz, 2H), 2.65 (t, J=4.8 Hz, 4H), 1.67 (p, J=6.6 Hz, 4H), 1.38-1.25 (m, 52H), 0.87 (t, J=6.4 Hz, 6H). ³¹P NMR (121 MHz, CDCl₃) δ 0.86.

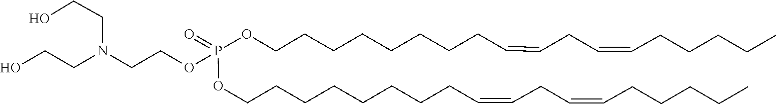 [SMILES: CCCCC/C=C\C/C=C\CCCCCCCCOP(=O)(OCCCCCCCC/C=C\C/C=C\CCCCC)OCCN(CCO)CCO]

ZYB20200617-1. ³¹P NMR (121 MHz, CDCl₃) δ 0.69. ¹H NMR (400 MHz, Chloroform-d) δ 5.45-5.25 (m, 8H), 4.14-4.00 (m, 6H), 3.59 (t, J=4.8 Hz, 4H), 2.77 (dd, J=7.8, 5.4 Hz, 6H), 2.70-2.61 (m, 4H), 2.04 (q, J=6.8 Hz, 8H), 1.67 (q, J=6.9 Hz, 4H), 1.39-1.21 (m, 32H), 0.89 (t, J=6.7 Hz, 6H).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A compound of Formula A: 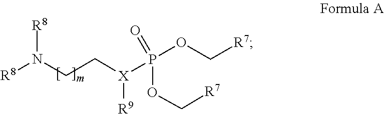
or a salt thereof,
wherein:
each R⁷ is independently selected from alkyl, alkenyl, alkynyl, ester, alkylester, alkylketal, alkylcarbonate, or alkylcarbamate;
each R⁸ is independently selected from alkyl, alkenyl, alkynyl, ester, alkylester, or alkylalcohol;
R⁹ is selected from hydrogen, alkyl, alkenyl, alkynyl, ester, alkylester, or 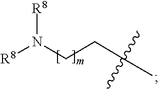
X is selected from O or N; and
m is an integer from 0 to 10.

2. The compound of claim 1, wherein each R⁸ is alkyl or alkylalcohol.

3. The compound of claim 2, wherein each R⁸ is methyl.

4. (canceled)

5. The compound of claim 1, wherein R⁹ is hydrogen or

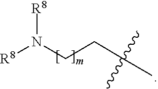

6. (canceled)

7. The compound of claim 1, wherein m is an integer from 1 to 3.

8. The compound of claim 1, wherein X is O.

9. The compound of claim 1, wherein X is N, and R⁹ is hydrogen or

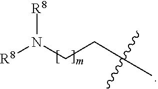

10. (canceled)

11. The compound of claim 1, wherein the compound has the formula: 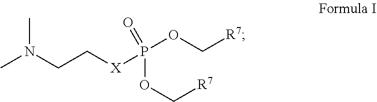
or a salt thereof,
wherein:
each R⁷ is independently selected from alkyl, alkenyl, alkynyl, ester, alkylester, alkylketal, alkylcarbonate, or alkylcarbamate; and
X is selected from O or N.

12. The compound of claim 1, wherein the compound has the formula: 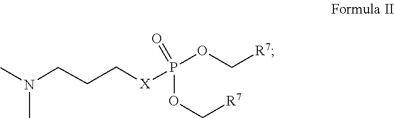
or a salt thereof,
wherein:
each R⁷ is independently selected from alkyl, alkenyl, alkynyl, ester, alkylester, alkylketal, alkylcarbonate, or alkylcarbamate; and
X is selected from O or N.

13. The compound of claim 1, wherein the compound has the formula: 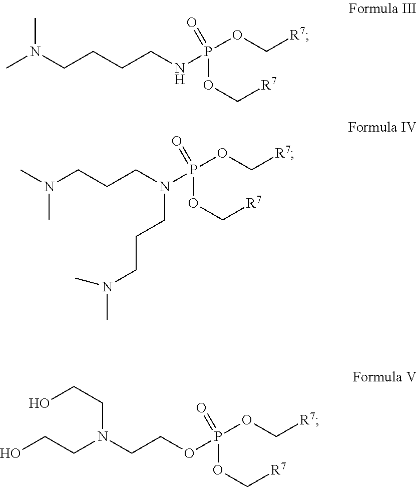
or a salt thereof,
wherein:
each R⁷ is independently selected from alkyl, alkenyl, alkynyl, ester, alkylester, alkylketal, alkylcarbonate, or alkylcarbamate.

14. (canceled)

15. (canceled)

16. The compound of claim 1, wherein each R⁷ is alkyl or alkenyl.

17. (canceled)

18. The compound of claim 1, wherein each R⁷ is independently selected from:

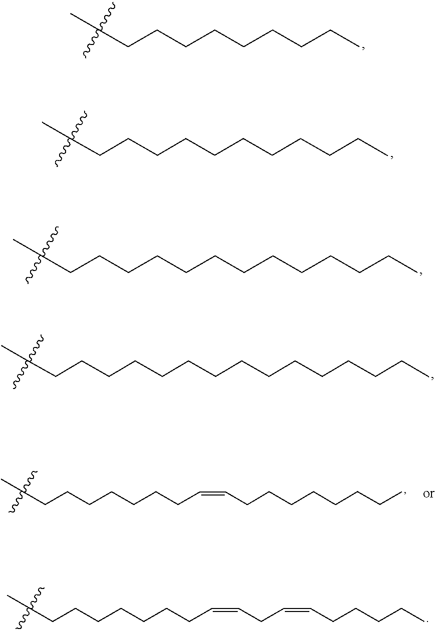

19. The compound of claim 1, wherein the compound has the formula: 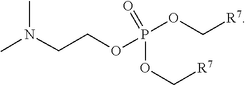
wherein each R⁷ is independently selected from:

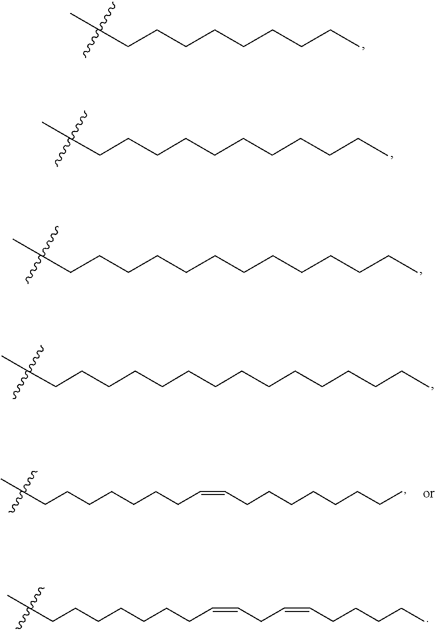

20. The compound of claim 1, wherein the compound has the formula: 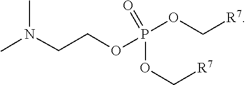
wherein each R⁷ is:

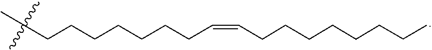 [SMILES: *CCCCCCC/C=C\CCCCCCCC]

21. The compound of claim 1, wherein the compound has the formula: 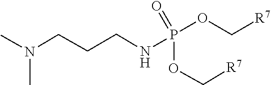
wherein each R⁷ is independently selected from:

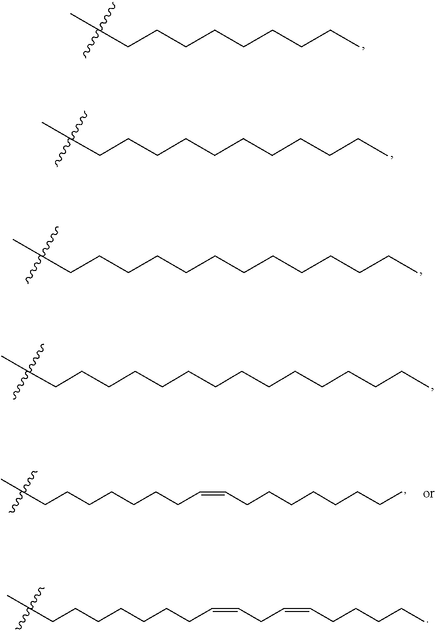

22. The compound of claim 1, wherein the compound has the formula: 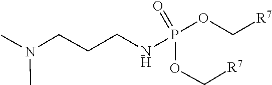
wherein each R⁷ is:

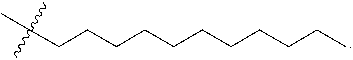 [SMILES: *CCCCCCCCCCC]

23. A composition comprising:
a compound of claim 1; and
an agent.

24. (canceled)

25. (canceled)

26. (canceled)

27. A nanoparticle comprising:
a compound of claim 1;
a non-cationic lipid;
a polyethylene glycol-lipid; and
a sterol.

28. (canceled)

29. (canceled)

30. (canceled)

31. (canceled)

32. A pharmaceutical composition comprising a pharmaceutically acceptable carrier and a therapeutically effective amount of a compound of claim 1.

33. A method for delivering an agent into a cell, comprising: introducing into the cell a composition of claim 23.
